(19) United States
(12) Patent Application Publication
(10) Pub. No.: US 2002/0020029 A1
(43) Pub. Date: Feb. 21, 2002

(54) Composition for the oxidation dyeing of keratinous fibers comprising at least one 1-(4-aminophenyl)pyrrolidine oxidation dye and at least one enzymatic oxidizing system, and dyeing methods

(76) Inventors: Sylvain Kravtchenko, Asnieres (FR); Gregory Plos, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 09/836,411

(22) Filed: Apr. 18, 2001

(30) Foreign Application Priority Data
Apr. 18, 2000 (FR) 00 04994

(51) Int. Cl. A61K 7/13

(52) U.S. Cl. 8/405; 8/406; 8/421

(57) ABSTRACT

Compositions for oxidation dyeing of keratinous fibers, for example, human keratinous fibers such as hair, comprising, in a medium suitable for dyeing, at least one enzymatic oxidizing system and at least one oxidation dye precursor chosen from the 1-(4-aminophenyl)pyrrolidine compounds and acid addition salts, of formula (I) and processes comprising such compositions.

The subject of the invention is a ready-to-use composition for the oxidation dyeing of keratinous fibers, such as human keratinous fibers, for example hair, comprising, in a medium suitable for dyeing, at least one oxidation dye precursor (oxidation base) chosen from the 1-(4-aminophenyl)pyrrolidines of formula (I), and at least one enzymatic oxidizing system comprising at least one enzyme chosen from 2 electron oxidoreductases, 4 electron oxidoreductases, and peroxidases, and dyeing methods using this composition.

It is known to dye keratinous fibers, and such as human hair, with dyeing compositions comprising oxidation dyes, such as, oxidation dye precursors (for example, ortho-phenylenediamines, para-phenylenediamines, ortho-aminophenols, para-aminophenols, and heterocyclic bases generally called oxidation bases). The oxidation dye precursors, or oxidation bases, are colorless or weakly colored compounds. When these compounds are combined with oxidizing products, dyes and colored compounds may form by a process of oxidative condensation.

It is also known that the shades obtained with these oxidation bases can be varied by combining them with couplers or color modifiers, the latter may be chosen from aromatic meta-diamines, meta-aminophenols, meta-diphenols and certain heterocyclic compounds.

The variety of molecules used in oxidation dyes (oxidation bases and couplers), allows a rich palette of colors to be obtained.

It is desirable for such oxidation dyes, otherwise called “permanent” dyes, to satisfy at least one of the following objectives: have no toxicological drawbacks, make it possible to obtain shades of the desired intensity and exhibit good resistance towards external agents for example, light, adverse weather conditions, washing, permanent waving, perspiration, and rubbing.

Further it is desirable that such dyes be able to cover grey hair, and thus should be the least selective possible. That is to say, the dyes should make it possible to obtain the smallest possible differences in color all along the same keratinous fiber, which may indeed be differently sensitized (i.e. damaged) between its tip and its root.

Oxidation dyeing of keratinous fibers is generally carried out in alkaline medium, in the presence of hydrogen peroxide. However, the use of alkaline media in the presence of hydrogen peroxide may possess the undesirable drawbacks of causing appreciable degradation of the fibers, as well as, considerable bleaching of the keratinous fibers.

Thus, dyeing hair permanently with para-phenylenediamine (PPD) coupling products in the presence of hydrogen peroxide is known. However, other types of oxidation bases have been sought and proposed as alternatives to PPD. A representative example of an alternative oxidation base used in commercial hair-dyeing products is the tertiary base N,N-bis(β-hydroxyethyl)-para-phenylenediamine.

Moreover, the oxidation dyeing of keratinous fibers may also be carried out with the aid of oxidizing systems, such as, enzymatic systems which are different from hydrogen peroxide. Dyeing keratinous fibers with compositions comprising at least one oxidation base and optionally at least one coupler, in combination with 2 electron oxidoreductases, such as, pyranose oxidase, glucose oxidase and uricase, in the presence of a donor for the said enzymes is disclosed in Patent Application EP-A-0 310 675, incorporated by reference herein.

Other references disclose dyeing keratinous fibers with compositions comprising an oxidation dye precursor and a laccase-type enzyme (4 electron oxidoreductases). These references, for example, include: Patent Applications FR-A-2112549, FR-A-2694018, EP-A-504005, WO95/07988, WO95/33836, WO95/33837, WO96/00290, WO97/19998, WO97/19999, WO97/37633, WO98/40471 and U.S. Pat. No. 3,251,742, all of which are incorporated by reference herein.

Other references, Patents EP-548620, BE-7751 10 and U.S. Pat. No. 3,893,803, discuss dyeing keratinous fibers using peroxidase in the presence of low quantities of hydrogen peroxide. These references are incorporated by reference herein.

These dyeing methods, although used under conditions which may diminish degradation of the keratinous fibers below the level of degradation found by the dyes used in the presence of hydrogen peroxide, nevertheless may lead to colors which are not yet completely satisfactory. For example, the processes may affect the depth of color.

However, the inventors have discovered that it is possible to obtain dyes capable of giving deep colors by combining at least one oxidation base chosen from 1-(4-aminophenyl)pyrrolidines of formula (I) with at least one enzymatic oxidizing system comprising at least one oxidoreductase-type enzyme. The at least one oxidoreductase-type enzyme may be chosen from 2 electron oxidoreductases, 4 electron oxidoreductases, and peroxidases.

The dyes according to the invention described above, make it possible to decrease the selectivity of the dye when compared to dyes obtained in compositions that combine a tertiary oxidation base, such as N,N-bis(β-hydroxyethyl)-para-phenylenediamine (widely used in commercial hair-dyeing products), with at least one enzyme.

One embodiment of the invention is a ready-to-use composition for oxidation dyeing of keratinous fibers, for example, human keratinous fibers such as hair, comprising, in a medium suitable for dyeing:

(1) at least one enzymatic oxidizing system comprising at least one enzyme chosen from 2 electron oxidoreductases, 4 electron oxidoreductases, and peroxidases; and

(2) at least one oxidation dye precursor chosen from 1-(4-aminophenyl)pyrrolidines of formula (I) and acid addition salts thereof:

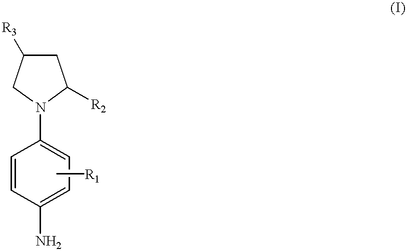

wherein:

R₁ is chosen from a hydrogen atom, (C₁-C₆)alkyl groups, (C₁-C₅)monohydroxyalkyl groups, and (C₂-C₅)polyhydroxyalkyl groups;

R₂ is chosen from a hydrogen atom, a —CONH₂ group, (C₁-C₅)monohydroxyalkyl groups, and (C₂-C₅)polyhydroxyalkyl groups;

R₃ is chosen from a hydrogen atom, and a hydroxyl group.

Non-limiting examples of acid addition salts are chosen from hydrochlorides, phosphates, hydrobromides, sulfates, tartrates, lactates and acetates.

Another embodiment of the invention is a method for the oxidation dyeing of keratinous fibers using at least one ready-to-use dyeing composition.

The expression “ready-to-use composition” is understood to mean, according to the purposes of the present invention, any composition intended to be applied immediately to the keratinous fibers, either stored as it is before use or obtained from the combining of two or more compositions.

The 1-(4-aminophenyl)pyrrolidines of formula (I) are known compounds with proposed uses for conventional oxidation hair dyeing with hydrogen peroxide. Examples of these compounds and uses are described in U.S. Pat. Nos. 5,851,237, 5,876,464 and 5,993,491, incorporated by reference herein.

In one embodiment of the invention, said 1-(4-aminophenyl)pyrrolidines of formula (I) are chosen from such compounds wherein: R₁, R₂ and R₃ are each a hydrogen atom. Thus the compound of formula (I) is then 1-(4-aminophenyl)pyrrolidine.

In another embodiment of the invention, said 1-(4-aminophenyl)pyrrolidines of formula (I) are chosen from such compounds wherein: R₁ and R₃ are each a hydrogen atom and R₂ is a —CH₂OH group. Thus, the compound of formula (I) is 1-(4-aminophenyl)-2-pyrrolidinemethanol.

In yet another embodiment of the invention, said 1-(4-aminophenyl)-pyrrolidines of formula (I) are chosen from such compounds wherein: R₁ is a hydrogen atom, R₂ is a —CH₂OH group and R₃ is a hydroxyl group. Thus the compound of formula (I) is 1 -(4-aminophenyl)-4-hydroxy-2-pyrrolidinemethanol.

In yet another embodiment of the invention, said 1-(4-aminophenyl)-pyrrolidines of formula (I) are chosen from such compounds wherein: R₁ and R₃ are each a hydrogen atom and R₂ is a —CONH₂ group. Thus the compound of formula (I) is N-(4-aminophenyl)prolineamide.

The at least one 1-(4-aminophenyl)pyrrolidines of formula (I) and acid addition salts thereof can be present in a composition according to the invention in an amount ranging, for example, from 0.001 to 10% by weight relative to the total weight of the composition in accordance with the invention such as, for example, 0.01 to 8% by weight relative to the total weight of the composition.

At Least One Enzymatic Oxidizing System

The at least one 2 electron oxidoreductase may be used in the presence of at least one corresponding donor for the corresponding enzymes. Examples of said at least one 2 electron oxidoreductase may be chosen from pyranose oxidases, glucose oxidases, glycerol oxidases, lactate oxidases, pyruvate oxidases, uricases, choline oxidases, sarcosine oxidases, bilirubin oxidases and amino acid oxidases.

In one embodiment of the invention, said at least one 2 electron oxidoreductase is chosen from the uricases of animal, microbiological or biotechnological origin.

Non-limiting examples of these uricases comprise: the uricase extracted from boar liver, the uricase from Arthrobacter globiformis and the uricase from Aspergillus flavus.

The at least one 2 electron oxidoreductase may be used in crystalline form or in a form diluted with a diluent inert in respect to said at least one 2 electron oxidoreductase.

Said at least one 2 electron oxidoreductase in accordance with the invention, may be present in an amount ranging, for example, from 0.01 to 20% by weight relative to the total weight of the composition, such as, from 0.1 to 5% by weight relative to the total weight of the composition.

It is also possible to define the quantity of at least one enzyme as a function of its activity. The enzymatic activity of said at least one 2 electron oxidoreductase may be defined from the oxidation of the corresponding at least one donor under aerobic conditions. One U unit corresponds to the quantity of said at least one 2 electron oxidoreductase leading to the generation of one pmol of H₂O₂ per minute at a pH of 8.5 and at a temperature of 25° C.

In one embodiment of the invention, the quantity of said at least one 2 electron oxidoreductase ranges between 10 and 108 U units per 100 g of dyeing composition.

According to the invention, the term “donor” means the various substrates which are necessary for the functioning of said at least enzyme.

The nature of said at least one donor (or substrate) for the corresponding enzyme varies according to the nature of said at least one 2 electron oxidoreductase in the dye composition of the invention. For example, in one embodiment, donors for the pyranose oxidases may be comprise D-glucose, L-sorbose and D-xylose; donors for the glucose oxidases may comprise D-glucose; donors for the glycerol oxidases may comprise glycerol and dihydroxyacetone; donors for the lactate oxidases may comprise lactic acid and its salts; donors for the pyruvate oxidases may comprise pyruvic acid and its salts; donors for the uricases may comprise uric acid and its salts; donors for the choline oxidases may comprise choline and its acid addition salts, such as, choline hydrochloride, and betaine aldehyde; donors for the sarcosine oxidases may comprise sarcosine, N-methyl-L-leucine, N-methyl-DL-alanine and N-methyl-DL-valine; and donors for the bilirubin oxidases may comprise bilirubin.

When utilized, said at least one donor (or substrate) may be present in a composition according to the invention, in an amount ranging from 0.01 to 20% by weight of the total weight of the composition, such as, from 0.1 to 5% of this weight.

Said at least one 4 electron oxidoreductase which may be present in the ready-to-use dyeing composition is chosen from laccases, tyrosinases, catechol oxidases and polyphenol oxidases.

In one embodiment of the invention, said at least one 4 electron oxidoreductase is chosen from laccases.

These laccases may be chosen from laccases of plant origin, laccases of animal origin, laccases of fungal origin (yeasts, moulds, fungi), laccases of bacterial origin, the organisms may be of mono- or pluricellular origin.

In another embodiment of the invention, said laccases may also be obtained by biotechnological techniques.

Non-limiting examples of laccases of plant origin may include the laccases produced by plants which perform chlorophyll synthesis, such as those indicated in Patent Application FR-A-2,694,018, incorporated by reference herein.

In one embodiment of the invention, said laccases may be present in plant extracts chosen from: Anacardiacea, such as, Magnifera indica, Schinus molle and Pleiogynium timoriense; Podocarpacea; Rosmarinus off.; Solanum tuberosum; Iris sp.; Coffea sp.; Daucus carrota; Vinca minor; Persea americana; Catharanthus roseus; Musa sp.; Malus pumila; Gingko biloba; Monotropa hypopithys (Indian pipe), Aesculus sp.; Acer pseudoplatanus; Prunus persica and Pistacia palaestina.

Examples of said laccases of fungal origin, optionally obtained by biotechnological techniques, which can be used according to the invention, may comprise laccases obtained from fungi chosen from Polyporus versicolor, Rhizoctonia praticola and Rhus vernicifera as described, for example, in patent applications FR-A-2 112 549 and EP-A-504 005 (incorporated by reference herein). Other examples of laccases obtained from fungal origin comprise the laccases described in patent applications WO 95/07988, WO 95/33836, WO 95/33837, WO 96/00290, WO 97/19998 and WO 97/19999, incorporated by reference herein, such as, the laccases obtained from: Scytalidium, Polyporus pinsitus, Myceliophthora thermophila, Rhizoctonia solani, Pyricularia orizae, and variants thereof.

Additional examples may also comprise the laccases obtained from: Trametes versicolor, Fomes fomentarius, Chaetomium thermophile, Neurospora crassa, Colorius versicol, Botrytis cinerea, Rigidoporus lignosus, Phellinus noxius, Pleurotus ostreatus, Aspergillus nidulans, Podospora anserina, Agaricus bisporus, Ganoderma lucidum, Glomerella cingulata, Lactarius piperatus, Russula delica, Heterobasidion annosum, Thelephora terrestris, Cladosporium cladosporioides, Cerrena unicolor, Coriolus hirsutus, Ceriporiopsis subvermispora, Coprinus cinereus, Panaeolus papilionaceus, Panaeolus sphinctrinus, Schizophyllum commune, Dichomitius squalens, and variants thereof.

In one embodiment of the invention said laccases of fungal origin are optionally obtained by biotechnological techniques.

In another embodiment of the invention, the enzymatic activity of said laccases comprising syringaldazine among their substrates may be defined by the oxidation of syringaldazine under aerobic conditions. One Lacu unit corresponds to the amount of enzyme which catalyses the conversion of 1 mmol of syringaldazine per minute at a pH of 5.5 and at a temperature of 30° C. One U unit corresponds to the amount of enzyme which produces an absorbance delta of 0.001 per minute at a wavelength of 530 nm, using syringaidazine as a substrate, at 30° C. and at a pH of 6.5.

Additionally, the enzymatic activity of said laccases used in accordance with the invention may also be defined by the oxidation of para-phenylenediamine. One Lacu unit corresponds to the amount of enzyme which produces an absorbance delta of 0.001 per minute at a wavelength of 496.5 nm, using para-phenylenediamine as substrate (64 mM), at 30° C. and at a pH of 5.

In another embodiment of the invention, the at least one 4-electron oxidoreductase is present in an amount ranging from 0.01 to 20% by weight relative to the total weight of the ready-to-use dye composition. In another embodiment, the at least one 4-electron oxidoreductase is present in an amount ranging from 0.1 to 5% by weight relative to the total weight of the ready-to-use dye composition.

When said laccases are used, the amount of said laccase present in the ready-to-use dye composition may vary as a function of the nature of the laccase used. In one embodiment of the invention, the laccase is present in the composition according to the invention in an amount ranging from 0.5 to 2000 Lacu (i.e. between 10,000 to 40×10⁶ U units or alternatively between 20 to 20×10⁶ Lacu units) per 100 g of ready-to-use dye composition.

In one embodiment of the invention, said at least one peroxidase which may be used as enzymatic oxidizing agent in the ready-to-use dye composition can be chosen from enzymes belonging to the subclass 1.11.1 described in the book Enzyme Nomenclature, Academic Press Inc., 1984 (incorporated by reference, herein). Some peroxidases belonging to this class require the presence of a donor to function. This is the case, for example, for the NADH peroxidases (1.11.1) having NADH as donor, the fatty acid peroxidases (1.11.1.3) having a fatty acid, such as, palmitate as donor, the NADPH peroxidases (1.11.1.2) having NADPH as donor, the cytochrome-c peroxidases (1.11.1.5) having ferrocytochrome-c as donor, the iodide peroxidases (1.11.1.8) having iodides as donor, the chloride peroxidases (1.11.10) having chlorides as donor, the L-ascorbate peroxidases (1.11.1.11) having L-ascorbate as donor and the glutathione peroxidases (1.11.1.9) having glutathione as donor.

Other non-limiting examples of said at least one peroxidase are those peroxidases belonging to the subclass 1.11.1 function without a donor other than the oxidation dye and are chosen from catalases (1.11.1.6) and simplex peroxidases (1.11.1.7).

In one embodiment of the invention, simplex peroxidases (1.11.1.7) are used.

The peroxidases function in the presence of hydrogen peroxide, which is provided in its native form or generated in situ via an enzymatic route by at least one 2-electron oxidoreductase and its corresponding at least one donor in the presence of air

The origin of said at least one peroxidase which may be used in the ready-to-use dye composition in accordance with the invention, is chosen from plant, animal, fungal and bacterial origin. In one embodiment of the invention, said at least one peroxidase may be obtained by biotechnological techniques.

Said at least one peroxidase may be obtained, for example, from the following sources: apple, apricot, barley, black radish, beetroot, cabbage, carrot, corn, cotton, garlic, grape, mint, rhubarb, soybean, spinach, inky cap, bovine milk and microorganisms, such as, Acetobacter peroxidans, Staphylococcus faecalis and Arthromyces ramosus.

The unit of activity of simplex peroxidase (1.11.1.7) can be defined as being the amount of simplex enzyme forming 1 mg of purpurogallin from pyrogallol in 20 s at pH 6 and at 20° C. By way of example, black radish peroxidase P6782 from Sigma® has an activity of about 250 units per mg.

In one embodiment of the invention, the working concentration of this type of enzyme ranges from 25 to 5×10⁶ units per 100 g of ready-to-use composition.

In one embodiment, said at least one peroxidase is present in the composition according to the invention in an amount ranging from 0.0001 to 20% by weight relative to the total weight of the composition, such as, 0.001 to 10% by weight relative to the total weight of the composition.

Couplers

The ready-to-use dyeing composition in accordance with the invention may comprise at least one coupler from those conventionally used in oxidation dyeing. Non-limiting examples of said at least one coupler is chosen from: meta-aminophenols, meta-phenylenediamines, meta-diphenols, naphthols and heterocyclic couplers, such as, for example, indole derivatives, indoline derivatives, sesamol and its derivatives, pyridine derivatives, pyrazolotriazole derivatives, pyrazolones, indazoles, benzimidazoles, benzothiazoles, benzoxazoles, 1,3-benzodioxoles, quinolines, and acid addition salts of any of the foregoing compounds.

In one embodiment of the invention said at least one coupler is chosen from: 2,4-diamino-1-(β-hydroxyethyloxy)benzene, 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2-amino-4-(β-hydroxyethylamino)-1 -methoxybenzene, 1,3-diaminobenzene, 1,3-bis(2,4-diaminophenoxy)propane, sesamol, 1-amino-2-methoxy-4,5-methylenedioxybenzene, α-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, 2-amino-3-hydroxypyridine, 3,6-dimethylpyrazolo[3,2-c]-1,2,4-triazole, 2,6-dimethylpyrazolo[1,5-b]-1,2,4-triazole, and acid addition salts of any of the foregoing compounds.

Said at least one coupler may be present in a composition according to the invention in an amount ranging, for example, from 0.0001 to 15% by weight of the total weight of the ready-to-use dyeing composition, such as, from 0.001 to 10% by weight.

Non-limiting examples of the acid addition salts of said at least one coupler optionally present in the dyeing compositions of the invention may be chosen from hydrochlorides, hydrobromides, sulfates, tartrates, lactates and acetates.

Additional Oxidation Bases

The ready-to-use dyeing composition in accordance with the invention may further comprise at least one additional oxidation base other than the at least one oxidation precursor of formula (I) according to the invention and normally used in oxidation dyeing chosen from may be chosen, for example, from para-phenylenediamine, para-tolylenediamine, 2-hydroxyethyl-para-phenylenediamine, 1-N,N-bis(2-hydroxyethyl)-para-phenylenediamine, para-aminophenols, such as, 3-methyl-4-aminophenol and 4-aminophenol, ortho-phenylene diamines, ortho-aminophenols, double bases, heterocyclic bases, such as, pyrimidines, such as, 2,4,5,6-tetraaminopyrimidine and pyrazoles, such as, I1-(2-hydroxyethyl)-4,5-diaminopyrazole, and acid addition salts of any of the foregoing compounds.

Said at least one additional oxidation base may be present in a composition according to the invention, in an amount ranging, for example, from 0.0001 to 15% by weight relative to the total weight of said composition.

Non-limiting examples of the acid addition salts of said at least one additional oxidation base optionally present in the dyeing compositions of the invention may be chosen from hydrochlorides, hydrobromides, sulfates, tartrates, lactates and acetates.

Direct Dyes

The ready-to-use dyeing composition in accordance with the invention may further comprise at least one direct dye which may modify the shades by enriching them with glints. Non-limiting examples of said at least one direct dye may be chosen from neutral, cationic, and anionic nitro dyes, cationic, and anionic azo dyes and cationic, and anionic anthraquinone dyes. The at least one direct dye may be present in a composition according to the invention in an amount ranging, for example, 0.001 to 20% relative to the total weight of the composition, such as, from 0.01 to 10% of the total weight of the composition.

Dyeing Medium

The medium (or carrier) suitable for dyeing the composition according to the invention can be, for example, an aqueous medium comprising water and may advantageously comprise at least one cosmetically acceptable organic solvent. The at least one cosmetically acceptable organic solvent may, for example, be chosen from glycerol, chosen from (C₁-C₄) alkanols, such as ethanol, and isopropanol, chosen from aromatic alcohols, such as benzyl alcohol and phenoxyethanol, chosen from glycols, such as propyleneglycol, and 2-butoxyethanol propyleneglycol, and ethers of glycols(for example, monomethyl, and monoethyl ethers of diethyleneglycol and for example, monomethyl ether of propyleneglycol), described in French Patent Application 2 773 475, incorporated by reference herein, and analogous products and mixtures thereof.

Said at least one cosmetically acceptable organic solvent may be present in the composition according to the invention in an amount ranging, for example, from 1 to 40% by weight relative to the total weight of the composition, such as, from 5 and 30% by weight relative to the total weight or the composition.

Generally, the pH of the ready-to-use composition is chosen such that the enzymatic activities of the 2 electron oxidoreductases, 4 electron oxidoreductases, and peroxidases are sufficient. The pH, for example, may range from 3 to 11, such as, for example, from 4 to 9. The pH may be adjusted to the desired value by means of at least one agent chosen from acidifying agents and alkalinizing agents normally utilized in dyeing keratinous fibers.

Representative acidifying agents include conventionally, by way of example, inorganic acids and organic acids such as hydrochloric acid, orthophosphoric acid and sulphuric acid, and carboxylic acids such as tartaric acid, citric acid, lactic acid and sulfonic acid.

Representative alkalinizing agents, may include, for example, aqueous ammonia, alkali metal carbonates, alkanolamines such as mono-, di- and triethanolamines and derivatives thereof, and 2-methyl-2-amino-1-propanol and derivatives thereof, sodium hydroxide, potassium hydroxide and compounds of formula (II):

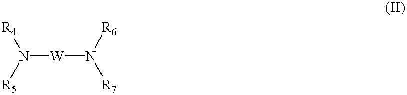

wherein:

W is a propylene residue optionally substituted with a group chosen from a hydroxyl group and (C₁-C₄)alkyl groups; and

R₄, R₅, R₆ and R₇, which are identical or different, are each chosen from a hydrogen atom, (C₁-C₄)alkyl groups and (C₁-C₄)hydroxyalkyl groups.

The ready-to-use dyeing composition in accordance with the invention may further comprise at least one adjuvant conventionally used in hair dyeing compositions, chosen from, for example, anionic, cationic, nonionic, amphoteric and zwitterionic surfactants, anionic, cationic, nonionic, amphoteric and zwitterionic polymers, inorganic and organic thickening agents, antioxidants, penetration-enhancing agents, sequestering agents, perfumes, buffers, dispersing agents, conditioning agents chosen from, for example, volatile and non-volatile, modified and nonmodified, silicones, examples of which are described in French Patent Application 2 773 474, incorporated by reference herein, film-forming agents, ceramides, preservatives, reducing agents and opacifying agents.

Among the organic thickening agents that may be employed in the invention are the chitosans described in French Patent Application FR-9907828 (incorporated by reference herein), non-limiting examples of which include chitosans chosen from:

chitosans salified with an organic acid, such as, for example, lactic acid, glutamic acid and pyrrolidonecarboxylic acid, and chitosans salified with an inorganic acid, such as, for example, hydrochloric acid and sulfuric acid;

chemically modified chitosans, such as, for example, the products chosen from: N,O-carboxymethylchitosan by the company CHITOGENICS LTD, carboxymethylchitosan sold under the name OLEVASAN by the company SINOLION, N-carboxybutylchitosan sold under the trade names CHITOLAM NB 101 and EVALSAN by the company CHITO BIOS, N-succinylchitosan sold under the name MEXOMERE PAD by the company CHIMEX, sold under the name KITINAMI by the company FRANCHITINE, and sold under the name SUCCINYL CHITOSAN by the company KATAKURA CHIKKARIN, and N-succinylcarboxymethylchitosan sold under the name CHITOSOLLEN by the company IKEDA.

The said chitosans are present in a composition according to the invention in an amount ranging, for example, from 0.01 to 20% by weight relative to the total weight of the ready-to-use dyeing composition, such as, from 0.1 to 5% by weight relative to the total weight of the ready-to-use dyeing composition.

Thickening Polymer Comprising at Least One Fatty Chain

The ready-to-use composition according to the invention may further comprise at least one thickening polymer comprising at least one fatty chain. Said at least one thickening polymer comprising at least one fatty chain may be chosen, for example, from (i) anionic, (ii) nonionic, and (iii) cationic type thickening polymers comprising at least one fatty chain.

In one embodiment of the invention, said thickening polymer comprising at least one fatty chain is present in the composition containing the 1-(4-aminophenyl)-pyrrolidine(s) of formula (I).

(i) Anionic Thickeners

Such anionic thickening polymers comprising at least one fatty chain can be chosen from:

(I) anionic polymers comprising at least one hydrophilic unit and at least one allyl ether unit comprising at least one fatty chain, for example said anionic polymers wherein said at least one hydrophilic unit comprises at least one ethylenic unsaturated anionic monomeric residue, such as vinylcarboxylic acid and further such as at least one monomeric residue chosen from acrylic acid and methacrylic acid residues, and wherein said at least one allyl ether unit comprising at least one fatty chain corresponds to the monomeric residue resulting from the monomer of formula (I):

CH₂═C—R′—CH₂—O—Bₙ—R (I)

wherein:

R′ is chosen from H and CH₃, B is chosen from ethyleneoxy groups, n is chosen from zero and integers ranging from 1 to 100, R is a hydrocarbon group chosen from alkyl, arylalkyl, aryl, alkylaryl and cycloalkyl groups, comprising from 8 to 30 carbon atoms, such as from 10 to 24, and further such as from 12 to 18 carbon atoms.

One embodiment of the invention comprises at least one allyl ether unit of the monomeric residue resulting from the monomer of formula (I), wherein R′ is H, n is equal to 10, and R is a stearyl (C₁₈) group.

Representative anionic amphiphilic polymers of this type are described and prepared, according to a method of emulsion polymerization, in patent EP-0,216,479, the disclosure of which is incorporated by reference herein.

As used herein, the term “lower alkyl” means an alkyl chosen from saturated and unsaturated, branched and unbranched C₁-C₆ alkyl groups.

Representative anionic thickening polymers comprising at least one fatty chain include for example polymers formed from 20% to 60% by weight of at least one monomer chosen from acrylic acid and methacrylic acid, 5% to 60% by weight of C₁-C₆ alkyl(meth)acrylates, 2% to 50% by weight of allyl ether comprising at least one fatty chain of formula (I), and up to 1% by weight of a crosslinking agent chosen from well known copolymerizable polyethylenic unsaturated monomers such as diallyl phthalate, allyl (meth)acrylate, divinylbenzene, (poly)ethylene glycol dimethacrylate and methylenebisacrylamide.

One embodiment could comprise at least one anionic thickening polymer chosen from crosslinked terpolymers of methacrylic acid, ethyl acrylate, and polyethylene glycol (10 EO) stearyl alcohol ether (Steareth 10), such as the products sold by the company ALLIED COLLOIDS under the names SALCARE SC 80 and SALCARE SC 90, which are aqueous emulsions comprising 30% of a crosslinked terpolymer of methacrylic acid, of ethyl acrylate and of steareth-10-allyl ether (40/50/10).

Anionic thickening polymers comprising at least one fatty chain can also be chosen from:

(II) anionic polymers comprising at least one hydrophilic unit of the olefinic unsaturated carboxylic acid type and at least one hydrophobic unit of the (C₁₀-C₃₀)alkyl ester of unsaturated carboxylic acid type.

Such polymers are chosen from polymers comprising:

(A) at least one hydrophilic unit formed from olefinic unsaturated carboxylic acid monomers of formula (II):

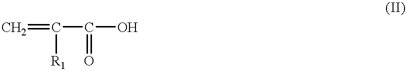

wherein:

R₁ is chosen from H, CH₃, and C₂H₅, (which corresponds with acrylic acid, methacrylic acid and ethacrylic acid units), and

(B) at least one hydrophobic unit formed from (C₁₀-C₃₀)alkyl esters of unsaturated carboxylic acid monomers of formula (III):

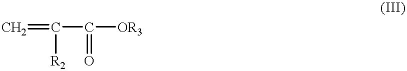

wherein:

R₂ is chosen from H, CH₃, and C₂H₅, (which corresponds with acrylate, methacrylate and ethacrylate units); and

R₃ is chosen from saturated and unsaturated, branched and unbranched (C₁₀-C₃₀) alkyl groups.

In one embodiment, for example, R₂ is chosen from H (acrylate units) and CH₃ (methacrylate units) and R₃ is chosen from (C₁₂-C₂₂)alkyl groups.

(C₁₀-C₃₀)alkyl esters of unsaturated carboxylic acids in accordance with the invention include for example lauryl acrylate, stearyl acrylate, decyl acrylate, isodecyl acrylate, dodecyl acrylate, lauryl methacrylate, stearyl methacrylate, decyl methacrylate, isodecyl methacrylate, and dodecyl methacrylate.

Anionic polymers of this type are for example described and prepared according to U.S. Pat. Nos. 3,915,921 and 4,509,949, the disclosures of which are incorporated by reference herein.

Anionic thickening polymers comprising at least one fatty chain that can be used include polymers formed from a mixture of monomers comprising:

(i) acrylic acid,

(ii) at least one ester of formula (III) described above wherein R₂ is chosen from H and CH₃, and R₃ is chosen from alkyl groups comprising from 12 to 22 carbon atoms, and

(iii) at least one crosslinking agent chosen from well known copolymerizable polyethylenic unsaturated monomers such as diallyl phthalate, allyl (meth)acrylate, divinylbenzene, (poly)ethylene glycol dimethacrylate and methylenebisacrylamide.

Representative anionic thickening polymers comprising at least one fatty chain that can be used include (i) polymers comprising 95% to 60% by weight of acrylic acid monomeric residue (hydrophilic unit), 4% to 40% by weight of C₁₀-C₃₀ alkyl acrylate monomeric residue (hydrophobic unit), and 0% to 6% by weight of crosslinking polymerizable monomeric residue, and (ii) polymers comprising 98% to 96% by weight of acrylic acid monomeric residue (hydrophilic unit), 1% to 4% by weight of C₁₀-C₃₀ alkyl acrylate monomeric residue (hydrophobic unit) and 0.1% to 0.6% by weight of crosslinking polymerizable monomeric residue such as those described above.

Among the above polymers, the products sold by the company GOODRICH under the trade names PEMULEN TR1, PEMULEN TR2, and CARBOPOL 1382 can be used. One embodiment could employ at least one polymer chosen from PEMULEN TR1, and the product sold by the company S.E.P.P.I.C. under the name COATEX SX.

Anionic thickening polymers comprising at least one fatty chain can also be chosen from:

(III) terpolymers formed from maleic anhydride/C30-C₃₈ α-olefin/alkyl maleate such as the product (maleic anhydride/C₃₀-C₃₈ α-olefin/isopropyl maleate copolymer) sold under the name PERFORMA V 1608 by the company NEWPHASE TECHNOLOGIES;

(IV) acrylic terpolymers formed from:

(a) 20% to 70% by weight of a carboxylic acid with α,β-monoethylenic unsaturation

(b) 20% to 80% by weight of a nonsurfactant monomer with α,β-monoethylenic unsaturation different from (a)

(c) 0.5% to 60% by weight of a nonionic monourethane which is the product of the reaction of a monohydric surfactant with a monoisocyanate with monoethylenic unsaturation

such as acrylic terpolymers described in patent application EP-A-0,173,109, the disclosure of which is incorporated by reference herein, and more particularly acrylic terpolymers described therein in Example 3, namely a methacrylic acid/methyl acrylate/dimethyl metaisopropenyl benzyl isocyanate of ethoxylated (40 EO) behenyl alcohol terpolymer in 25% aqueous dispersion;

(V) copolymers formed from at least two monomers, wherein at least one of said at least two monomers is chosen from a carboxylic acid with α,β-monoethylenic unsaturation, an ester of a carboxylic acid with α,β-monoethylenic unsaturation, and an oxyalkylenated fatty alcohol; and

(VI) copolymers formed from at least three monomers, wherein at least one of said at least three monomers is chosen from a carboxylic acid with, α,β-monoethylenic unsaturation, at least one of said at least three monomers is chosen from an ester of a carboxylic acid with monoethylenic unsaturation and at least one of said at least three monomers is chosen from an oxyalkylenated fatty alcohol.

Additionally, these compounds can also comprise, as monomer, a carboxylic acid ester comprising an α,β-monoethylenic unsaturation and a C₁-C₄ alcohol. By way of example of this type of compound, there may be mentioned ACULYN 22 sold by the company ROHM and HAAS, which is an oxyalkylenated stearyl methacrylate/ethyl acrylate/methacrylic acid terpolymer.

(ii) Nonionic Thickeners

Nonionic thickening polymers comprising at least one fatty chain according to the invention can be chosen from:

(1) celluloses modified by at least one group comprising at least one fatty chain such as:

hydroxyethylcelluloses modified by at least one group comprising at least one fatty chain such as alkyl, arylalkyl and alkylaryl groups and further such as alkyl, arylalkyl and alkylaryl groups wherein said alkyl groups comprise from 8-22 carbon atoms, such as the product NATROSOL PLUS GRADE 330 CS (C₁₆ alkyls) sold by the company AQUALON, and the product BERMOCOLL EHM 100 sold by the company BEROL NOBEL,

hydroxyethylcelluloses modified by at least one polyalkylene glycol ether of alkylphenol group, such as the product AMERCELL POLYMER HM-1500 (polyethylene glycol (15) ether of nonylphenol) sold by the company AMERCHOL;

(2) hydroxypropylguars modified by at least one group comprising at least one fatty chain such as the product ESAFLOR HM 22 (C₂₂ alkyl chain) sold by the company LAMBERTI, the products RE 210-18 (C₁₄ alkyl chain) and RE 205-1 (C₂₀ alkyl chain) sold by the company RHONE POULENC (Succeeded by RHODIA CHIMIE);

(3) copolymers formed from vinylpyrrolidone and at least one hydrophobic monomer comprising at least one fatty chain such as for example:

the products ANTARON V216 and GANEX V216 (vinylpyrrolidone/hexadecene copolymer) sold by the company I.S.P., and

the products ANTARON V220 and GANEX V220 (vinylpyrrolidone/eicosene copolymer) sold by the company I.S.P.;

(4) copolymers formed from at least one C₁-C₆ alkyl methacrylate and at least one amphiphilic monomer comprising at least one fatty chain and copolymers formed from at least one C₁-C₆ alkyl acrylate and at least one amphiphilic monomer comprising at least one fatty chain such as for example the oxyethylenated stearyl acrylate/methyl acrylate copolymer sold by the company GOLDSCHMIDT under the name ANTIL 208;

(5) copolymers formed from at least one hydrophilic methacrylate and at least one hydrophobic monomer comprising at least one fatty chain and copolymers formed from at least one hydrophilic acrylate and at least one hydrophobic monomer comprising at least one fatty chain such as for example the polyethylene glycol methacrylate/lauryl methacrylate copolymer;

(6) polyether-polyurethanes comprising in their chain both hydrophilic sequences which are most often of a polyoxyethylenated nature and hydrophobic sequences which may be chains chosen from aliphatic chains, cycloaliphatic chains, and aromatic chains; and

(7) polymers comprising an aminoplast ether backbone possessing at least one fatty chain, such as the compounds PURE THIX provided by the company SUD-CHEMIE.

Nonionic thickening polymers can additionally include polyether-polyurethanes comprising at least two lipophilic (i.e., hydrophobic) hydrocarbon chains, comprising from 6 to 30 carbon atoms, separated by a hydrophilic sequence, it being possible for the hydrocarbon chains to be chosen from pendant chains and chains at the end of a hydrophilic sequence. One embodiment may comprise at least one pendant chain. In addition, the polymer may comprise a hydrocarbon chain at least one end of a hydrophilic sequence.

Representative polyether-polyurethanes useful in the present invention may be polyblocks, such as in triblock form. The hydrophobic sequences may be at each end of the chain (for example: triblock copolymer with hydrophilic central sequence) and optionally both at the ends and in the chain (polyblock copolymer for example). These same polymers may also be in the form of graft units or may be star-shaped.

The nonionic polyether-polyurethanes comprising at least one fatty chain may be triblock copolymers whose hydrophilic sequence is a polyoxyethylenated chain comprising from 50 to 1000 oxyethylenated groups. Certain nonionic polyether-polyurethanes comprise a urethane bond between the hydrophilic sequences.

By extension, those whose hydrophilic sequences are linked by other chemical bonds to the lipophilic sequences are also included among the nonionic polyether-polyurethanes comprising at least one fatty chain.

Representative nonionic polyether-polyurethanes comprising at least one fatty chain include Rhéolate 205 comprising a urea function sold by the company RHEOX and Rhéolate 208, 204 and 212, as well as Acrysol RM 184, Aculyn 44 and Aculyn 46 from the company ROHM and HMS [ACULYN 46 is a polycondensate of polyethylene glycol comprising 150 or 180 mol of ethylene oxide, stearyl alcohol and methylene-bis(4-cyclohexylisocyanate) (SMDI), at 15% by weight in a maltodextrin (4%) and water (81%) matrix; ACULYN 44 is a polycondensate of polyethylene glycol comprising 150 or 180 mol of ethylene oxide, decyl alcohol and methylenebis(4-cyclohexylisocyanate) (SMDI), at 35% by weight in a propylene glycol (39%) and water (26%) mixture].

There may also be mentioned the product ELFACOS T210 comprising a C₁₂-C₁₄ alkyl chain and the product ELFACOS T212 comprising a C₁₈ alkyl chain from AKZO.

The product DW 1206B from RHOM & HMS comprising a C₂₀ alkyl chain and with a urethane bond, sold at 20% dry matter content in water, may also be used.

It is also possible to use solutions and dispersions of these polymers for example in water and for example in an aqueous-alcoholic medium. By way of example of such polymers, there may be mentioned Rhéolate 255, Rhéolate 278 and Rhéolate 244 sold by the company RHEOX. It is also possible to use the product DW 1206F and DW 1206J provided by the company ROHM & HMS.

Representative polyether-polyurethanes that can be used according to the invention include polyether-polyurethanes described in the article by G. Fonnum, J. Bakke and Fk. Hansen—Colloid Polym. Sci 271, 380, 389 (1993), the disclosure of which is incorporated by reference herein.

(iii) Cationic Thickeners

As used herein, “cationic thickener” refers to polymers chosen from polymers comprising at least one cationic group and polymers comprising at least one group which can be ionized to form cationic groups.

Representative cationic thickening polymers comprising at least one fatty chain used in the present invention can be chosen from quaternized cellulose derivatives and polyacrylates with noncyclic amine-containing side groups.

Such quaternized cellulose derivatives can be chosen from:

quaternized celluloses modified by groups comprising at least one fatty chain, such as at least one group chosen from alkyl, arylalkyl and alkylaryl groups comprising at least 8 carbon atoms,

quaternized hydroxyethylcelluloses modified by at least one group comprising at least one fatty chain, such as at least one group chosen from alkyl, arylalkyl and alkylaryl groups comprising at least 8 carbon atoms.

In one embodiment, said alkyl groups carried by the above quaternized celluloses and hydroxyethylcelluloses comprise from 8 to 30 carbon atoms and the aryl groups are chosen from phenyl, benzyl, naphthyl and anthryl groups.

There may be mentioned as examples of quaternized alkylhydroxyethylcelluloses comprising at least one C₈-C₃₀ fatty chain the products QUATRISOFT LM 200, QUATRISOFT LM-X 529-18-A, QUATRISOFT LM-X 529-18B (C₁₂ alkyl) and QUATRISOFT LM-X 529-8 (C₁₈ alkyl) marketed by the company AMERCHOL and the products CRODACEL QM, CRODACEL QL (C₁₂ alkyl) and CRODACEL QS (C₁₈ alkyl) marketed by the company CRODA.

Representative polyacrylates with amine-containing side groups, quaternized and otherwise, comprise for example hydrophobic groups of the steareth 20 type (polyoxyethylenated stearyl alcohol (20)).

As examples of polyacrylates with amine-containing side groups, there may be mentioned the polymers 8781-121B or 9492-103 from the company NATIONAL STARCH.

One embodiment of the oxidation dyeing composition according to the invention may comprise at least one nonionic thickening polymer comprising at least one fatty chain.

The anionic, nonionic and cationic at least one thickening polymer comprising at least one fatty chain is generally present in an amount ranging for example from 0.01% to 10% by weight relative to the total weight of the dyeing composition, such as from 0.1% to 5% by weight relative to the total weight of the dyeing composition.

Cationic Polymers

As used herein, “cationic polymer” refers to polymers chosen from polymers comprising at least one cationic group and polymers comprising at least one group which can be ionized to form cationic groups.

Representative cationic polymers which may be used in accordance with the present invention include any of those already known to improve at least one cosmetic property of hair, such as, for example, those described in patent application EP-A-0 337 354 and in French patent applications FR-A-2 270 846, 2 383 660, 2 598 611, 2 470 596 and 2 519 863, the disclosures of which are incorporated herein by reference.

According to the present invention, the at least one cationic polymer may be chosen from polymers comprising at least one unit, wherein said at least one unit comprises at least one group chosen from primary amine groups, secondary amine groups, tertiary amine groups and quaternary amine groups, wherein said at least one group forms part of the polymer skeleton, or is carried by at least one lateral substituent on said polymer skeleton.

According to the present invention, the at least one cationic polymer has a number-average molecular mass generally ranging for example from 500 to 5×10⁶, such as from 1×10³ to 3×10⁶.

The at least one cationic polymer may, for example, be chosen from polymers of quaternary polyammonium type, polymers of polyamino amide type and polymers of polyamine type. Such types of polymers are known in the art. They are for example described in French patents Nos. 2, 505, 348 and 2, 542, 997, the disclosures of which are incorporated by reference herein.

Non-limiting examples of cationic polymers include:

(1) homo- and co-polymers derived from at least one monomer chosen from acrylic esters, methacrylic esters and amides, wherein said homo- and co-polymers comprise at least one unit chosen from units of formulae: (I), (II), (III) and (IV):

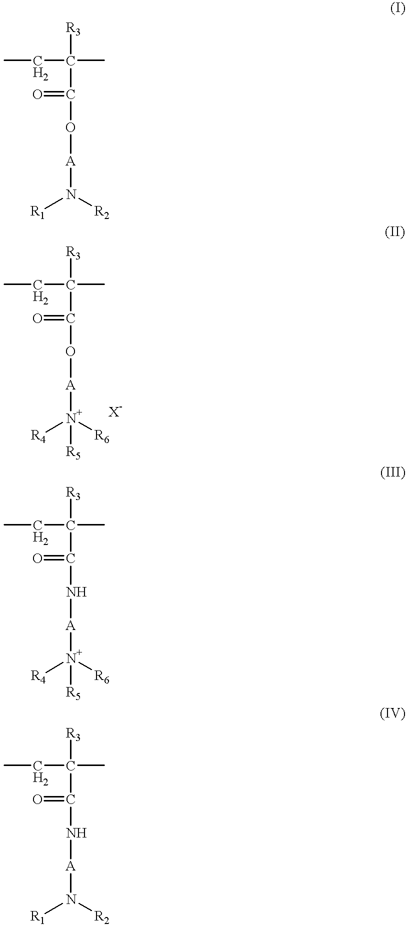

wherein:

R₃, which may be identical or different, are each chosen from a hydrogen atom and a methyl group;

A, which may be identical or different, are each chosen from linear and branched (C₁-C₆)alkyl groups, such as,(C₂-C₃)alkyl groups, and (C₁-C₄)hydroxyalkyl groups;

R₄, R₅ and R₆, which may be identical or different, are each chosen from (C₁-C₁₈)alkyl groups, such as, (C₁-C₆), and a benzyl group;

R₁ and R₂, which may be identical or different, are each chosen from a hydrogen atom and (C₁-C₆)alkyl groups, such as, a methyl group and an ethyl group;

X⁻ is an anion chosen from anions derived from at least one inorganic acid and anions derived from at least one organic acid, such as a methylsulfate anion and halides, such as a chloride and a bromide.

(1) homo- and Copolymers of family (1) may further comprise at least one unit derived from at least one comonomer chosen from vinyllactams, vinyl esters, acrylamides, methacrylamides, diacetone acrylamides, acrylamides and methacrylamides substituted on the nitrogen with at least one group chosen from (C₁-C₄) alkyls, acrylic acids, methacrylic acids, acrylic esters, and methacrylic esters. Non-limiting examples of vinyllactams include vinylpyrrolidone and vinylcaprolactam.

Non-limiting examples of copolymers of family (1) include:

copolymers derived from at least one monomer of (i) acrylamide and (ii) dimethylaminoethyl methacrylate quaternized with at least one group chosen from a dimethylsulfate group and dimethylhalides, such as the product sold under the name HERCOFLOC by the company Hercules;

copolymers derived from at least one monomer of (i) acrylamide and (ii) methacryloyloxy-ethyltrimethylammonium chloride described, for example, in patent application EP-A-080 976, the disclosure of which is incorporated herein by reference, and which is sold under the name BINA QUAT P 100 by the company Ciba Geigy;

copolymers derived from at least one monomer of (i) acrylamide and (ii) methacryloyloxy-ethyltrimethylammonium methosulfate, such as, for example, copolymers sold under the name RETEN by the company Hercules;

quaternized and non-quaternized vinylpyrrolidone/dialkylaminoalkyl acrylate copolymers and quaternized and non-quaternized vinylpyrrolidone/dialkylaminoalkyl methacrylate copolymers, such as the products sold under the name “GAFQUAT” by the company ISP, such as, for example, “GAFQUAT 734” or “GAFQUAT 755” and the products known as “COPOLYMER 845, 958 and 937”. These polymers are described in detail in French patents 2 077 143 and 2 393 573, the disclosures of which are incorporated herein by reference;

dimethylaminoethyl methacrylate/vinylcaprolactam/vinylpyrrolidone terpolymers, such as the product sold under the name GAFFIX VC 713 by the company ISP;

vinylpyrrolidone/methacrylamidopropyldimethylamine copolymers, such as the product sold under the name STYLEZE CC 10 by ISP; and

quaternized vinylpyrrolidone/dimethylaminopropylmethacrylamide copolymers, such as the product sold under the name “GAFQUAT HS 100” by the company ISP;

(2) cellulose ether derivatives comprising quaternary ammonium groups, such as those described in French patent 1,492,597, the disclosure of which is incorporated herein by reference, and polymers sold under the names “JR” (JR 400, JR 125 and JR 30M) and “LR” (LR 400, or LR 30M) by the company Union Carbide Corporation. These polymers are also defined in the CTFA dictionary as quaternary ammoniums of hydroxyethylcellulose which have reacted with an epoxide substituted with a trimethylammonium group;

(3) cationic cellulose derivatives such as cellulose copolymers and cellulose derivatives grafted with at least one water-soluble monomer of quaternary ammonium, such as those described in U.S. Pat. No. 4,131,576, the disclosure of which is incorporated herein by reference, such as hydroxyalkylcelluloses (such as, for example, hydroxymethylcelluloses, hydroxyethylcelluloses and hydroxypropylcelluloses, wherein said hydroxyalkylcelluloses are grafted with at least one salt chosen from, for example, methacryloylethyltrimethylammonium salts, methacrylamidopropyltrimethylammonium salts and dimethyidiallylammonium salts). For example, commercial products corresponding to the aforementioned cationic cellulose derivatives include the products sold under the names “CELQUAT L 200” and “CELQUAT H 100” by the company National Starch;

(4) cationic polysaccharides, such as those described in U.S. Pat. Nos. 3,589,578 and 4,031,307, the disclosures of which are incorporated herein by reference, such as guar gums comprising at least one cationic trialkylammonium group. For example, guar gums modified with at least one salt, such as a chloride salt, of 2,3-epoxypropyltrimethylammonium may be used in the present invention. Such products are sold in particular under the trade names JAGUAR C13 S, JAGUAR C 15, JAGUAR C 17 and JAGUAR C162 by the company Meyhall;

(5) polymers comprising (i) at least one piperazinyl unit and (ii) at least one group chosen from divalent alkylene groups and divalent hydroxyalkylene groups, wherein said at least one group optionally comprises at least one chain chosen from straight chains and branched chains, wherein said at least one chain is optionally interrupted by at least one entity chosen from an oxygen atom, a sulfur atom, a nitrogen atom, aromatic rings and heterocyclic rings, the oxidation products of said polymers and the quaternization products of said polymers. For example, such polymers are described in French patents 2,162,025 and 2,280,361, the disclosures of which are incorporated herein by reference;

(6) water-soluble polyamino amides which may be prepared by at least one polycondensation reaction of at least one acidic compound and at least one polyamine compound, wherein said polyamino amides may be crosslinked with at least one crosslinking agent chosen from epihalohydrins, diepoxides, dianhydrides, unsaturated dianhydrides, bis-unsaturated derivatives, bis-halohydrins, bis-azetidiniums, bis-haloacyidiamines, bis-alkyl halides and oligomers derived from reaction of at least one difunctional compound with at least one compound chosen from bis-halohydrins, bis-azetidiniums, bis-haloacyldiamines, bis-alkyl halides, epihalohydrins, diepoxides and bis-unsaturated derivatives, wherein said crosslinking agent may be used in a proportion generally ranging from 0.025 mol to 0.35 mol per amine group of said polyamino amide, wherein said polyamino amides may optionally be alkylated, and wherein if said polyamino amides comprise at least one tertiary amine group, said polyamino amides may optionally be quaternized. For example, such polymers are described in French patents 2,252,840 and 2,368,508, the disclosures of which are incorporated herein by reference;

(7) polyamino amide derivatives derived from condensation of at least one polyalkylene polyamine with at least one polycarboxylic acid, followed by alkylation with at least one bifunctional agent. Non-limiting examples of such polyamino amide derivatives include adipic acid/dialkylaminohydroxyalkyidialkylenetriamine polymers wherein said alkyl group is chosen from (C₁-C₄)alkyl groups, such as a methyl group, an ethyl group and a propyl group. For example, such polymers are described in French patent 1,583,363, the disclosure of which is incorporated herein by reference.

Other non-limiting examples of such derivatives include the adipic acid/dimethylaminohydroxypropyl/diethylenetriamine polymers sold under the name “CARTARETINE F, F4 or F8” by the company Sandoz.

(8) polymers derived from the reaction of (i) at least one polyalkylene polyamine comprising two primary amine groups and at least one secondary amine group with (ii) at least one dicarboxylic acid chosen from diglycolic acid and saturated aliphatic dicarboxylic acids comprising from 3 to 8 carbon atoms. According to the present invention, the molar ratio of the at least one polyalkylene polyamine to the at least one dicarboxylic acid generally ranges from 0.8:1 to 1.4:1. The polyamino amide resulting from the above reaction may be reacted with epichlorohydrin in a molar ratio of epichlorohydrin to the at least one secondary amine group of the polyamino amide generally ranging from 0.5:1 to 1.8:1. For example, such polymers are described in U.S. Pat. Nos. 3,227,615 and 2,961,347, the disclosures of which are incorporated herein by reference.

Polymers of this type are sold in particular under the name “HERCOSETT 57” by the company Hercules Inc. and under the name “PD 170” or “DELSETTE 101” by the company Hercules in the case of adipic acid/epoxypropyl/diethylenetriamine copolymers.

(9) cyclopolymers of alkyldiallylamine and cyclopolymers of dialkyldiallylammonium, such as homopolymers and copolymers comprising, as a constituent of the chain, at least one unit chosen from units of formulae (V) and (VI):

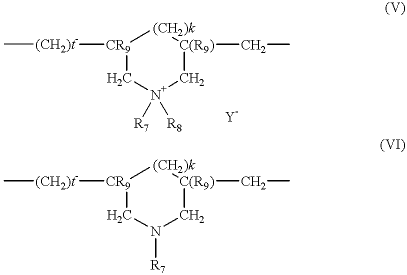

wherein:

k and t, which may be identical or different, are each chosen from 0 and 1, with the proviso that the sum of k+t is equal to 1;

R₉, which may be identical or different, are each chosen from a hydrogen atom and a methyl group;

R₇ and R₈, which may be identical or different, are each chosen from alkyl groups comprising from (C₁-C₂₂)alkyl groups, such as, (C₁-C₄)alkyl groups, hydroxyalkyl groups, such as (C₁-C₅)hydroxyalkyl groups, and (C₁-C₄)amidoalkyl groups;

R₇ and R₈, together with the nitrogen cation to which they are commonly bonded, may additionally form at least one cationic heterocyclic group, such as a cationic piperidyl group and a cationic morpholinyl group;

Y⁻ is an anion, such as a bromide anion, a chloride anion, an acetate anion, a borate anion, a citrate anion, a tartrate anion, a bisulfate anion, a bisulfite anion, a sulfate anion and a phosphate anion. For example, such polymers are described in French patent 2,080,759 and in its Certificate of Addition 2,190,406, the disclosures of which are incorporated herein by reference.

(9) cyclopolymers Non-limiting examples of the polymers defined above include the dimethyidiallylammonium chloride homopolymer sold under the name “MERQUAT 100” by the company Calgon (and its homologues of low weight-average molecular mass) and copolymers of diallyidimethylammonium chloride and of acrylamide, sold under the name “MERQUAT 550”.

(10) quaternary diammonium polymers comprising repeating units of formula (Vl):

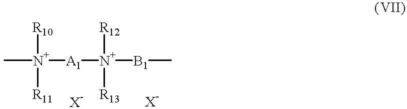

wherein:

R₁₀, R₁₁, R₁₂ and R₁₃, which may be identical or different, are each chosen from (C₁-C₂₀)aliphatic, (C₂-C₂₂)alicyclic groups, (C₅-C₂₀)arylaliphatic groups, and lower hydroxyalkyl groups; and

additionally at least two of said R₁₀, R₁₁, R₁₂ and R₁₃,

together with the nitrogen cations to which they are attached, may form at least one cationic heterocycle optionally comprising an additional heteroatom other than nitrogen; and

additionally, R₁₀, R₁₁, R₁₂ and R₁₃, which may be identical or different, are each chosen from linear and branched (C₁-C₆)alkyl groups substituted with at least one group chosen from a nitrile group, ester groups, acyl groups, amide groups and groups chosen from groups of formulae —CO—O—R₁₇—D and —CO—NH—R₁₇—D wherein R₁₇ is chosen from alkylene groups and D is chosen from quaternary ammonium groups;

A₁ and B₁, which may be identical or different, are each chosen from linear and branched, saturated and unsaturated (C₂-C₂₀)polymethylene groups, wherein said polymethylene groups may optionally comprise, optionally linked to and optionally intercalated in the main chain, at least one entity chosen from aromatic rings, oxygen atoms, sulfur atoms, sulfoxide groups, sulfone groups, disulfide groups, amino groups, alkylamino groups, hydroxyl groups, quaternary ammonium groups, ureido groups, amide groups and ester groups; and

X⁻ is an anion chosen from anions derived from inorganic acids and anions derived from organic acids; and

A₁, R₁₀ and R₁₂ may optionally form, together with the nitrogen atoms to which they are attached, at least one piperazine ring;

with the proviso that if A₁ is chosen from linear and branched, saturated and unsaturated (C₂-C₂₀)polymethylene groups and linear and branched, saturated and unsaturated hydroxy(C₂-C₂₀)polymethylene groups, B₁ may also be chosen from groups of formula:

—(CH₂)ₙ—CO—D—OC—(CH₂)ₙ—

wherein:

n is a number between 1 and 100,such as, 1 and 50;

D is chosen from:

a) glycol residues of formula: —O—Z—O—, wherein Z is chosen from linear and branched hydrocarbon groups and groups chosen from groups of formulae:

—(CH₂—CH₂—O)ₓ—CH₂—CH₂—; and

—[CH₂—CH(CH₃)—O]_{y}—CH₂—CH(CH₃)—;

wherein x and y, which may be identical or different, are each chosen from integers ranging from 1 to 4 (in which case x and y represent a defined and unique degree of polymerization) and any number ranging from 1 to 4 (in which case x and y represent an average degree of polymerization);

b) bis-secondary diamine residues such as piperazine derivatives;

c) bis-primary diamine residues chosen from residues of formula: —NH—Y—NH—, wherein Y is chosen from linear and branched hydrocarbon groups and residues of formula —CH₂—CH₂—S—S—CH₂—CH₂—; and

d) ureylene groups of formula: —NH—CO—NH—.

In one embodiment, X is an anion chosen from a chloride anion and a bromide anion.

According to the present invention, the quarternary diammonium polymers have a number-average molecular mass generally ranging, for example, from 1000 to 100,000.

For example, polymers of this type are described in French Patent Nos. 2,320,330, 2,270,846, 2,316,271, 2,336,434 and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653,4,026,945 and 4,027,020, the disclosures of which are incorporated herein by reference.

Further, according to the present invention, polymers comprising repeating units of formula (VIII) may be used:

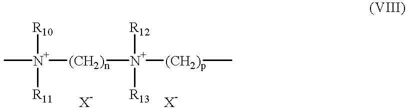

wherein:

R₁₀, R₁₁, R₁₂ and R₁₃, which may be identical or different, are each chosen from (C₁-C₄)alkyl groups and (C₁-C₄)hydroxyalkyl groups;

n and p, which may be identical or different, are each chosen from integers ranging from 2 to 20; and

X⁻ is an anion chosen from anions derived from inorganic acids and anions derived from organic acids.

(11) polyquaternary ammonium polymers comprising repeating units of formula (IX):

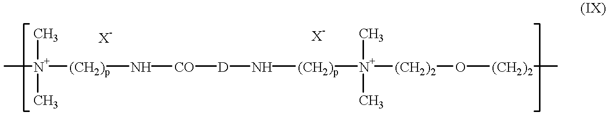

wherein:

p is an integer ranging from 1 to 6,

D is chosen from a direct bond and —(CH₂)ᵣCO— groups, wherein r is a number equal to 4 or 7, and

X⁻ is an anion chosen from anions derived from inorganic acids and anions derived from organic acids.

For example, such compounds are described in patent application EP-A-122,324, the disclosure of which is incorporated by reference herein, and may be prepared according to the procedures described in the U.S. Pat. Nos. 4,157,388, 4,390,689, 4,702,906, and 4,719,282, the disclosures of which are incorporated by reference herein.

Among these, there may be mentioned for example the products “Mirapol A 15”, “Mirapol AD1”, “Mirapol AZ1” and “Mirapol 175” sold by the company Miranol.

(12) quaternary polymers of vinylpyrrolidone and quaternary polymers of vinylimidazole, such as, for example, the products sold under the names LUVIQUAT FC 905, FC 550 and FC 370 by the company BASF.

(13) polyamines, such as POLYQUART H sold by Henkel under the reference name “POLYETHYLENE GLYCOL (15) TALLOW POLYAMINE” in the CTFA dictionary.

(14) crosslinked (meth)acryloyloxy(C₁-C₄)alkyltri(C₁-C₄)alkylammonium salt polymers, such as the polymers derived from homopolymerization of dimethylaminoethyl methacrylate quaternized with methyl chloride and polymers derived from copolymerization, for example, of acrylamide with dimethylaminoethyl methacrylate quaternized with a methyl halide (such as methyl chloride), wherein the homo- or copolymerization is followed by crosslinking with at least one compound comprising olefinic unsaturation, such as methylenebisacrylamide. For example, a crosslinked acrylamide/methacryloyloxyethyltrimethylammonium chloride copolymer (20/80 by weight) in the form of a dispersion comprising about 50% by weight of said copolymer in mineral oil may be used. This dispersion is sold under the name “SALCARE SC 92” by the company Allied Colloids. Further, a crosslinked methacryloyloxyethyltrimethylammonium chloride homopolymer comprising about 50% by weight of the homopolymer in mineral oil or in a liquid ester may be used. These dispersions are sold under the names “SALCARE SC 95” and “SALCARE SC 96” by the company Allied Colloids.

The at least one cationic polymer according to the present invention, may for example, be chosen from polyalkyleneimines (such as polyethyleneimines), polymers comprising at least one vinylpyridine unit, polymers comprising at least one vinylpyridinium unit, condensates of polyamines, condensates of epichlorohydrin, quaternary polyureylenes and chitin derivatives.

Other cationic polymers which may be used as the at least one cationic polymer according to the present invention are polyalkyleneimines (such as polyethyleneimines), polymers comprising at least one vinylpyridine unit, polymers comprising at least one vinylpyridinium unit, condensates of polyamines, condensates of epichlorohydrin, quaternary polyureylenes and chitin derivatives.

Other embodiments of the invention use cationic polymers chosen from the polymers of (1), (9), (10), (11) and (14). Specifically, polymers of formulae (W) and (U) can be used:

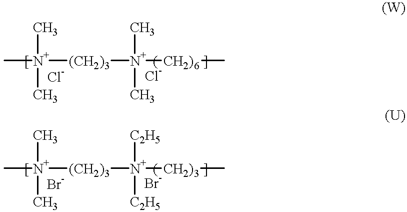

Amphoteric Polymers

The amphoteric polymers which can be used in the present invention can be chosen from polymers comprising K and M units distributed statistically in the polymer chain, wherein:

K is a unit derived from a monomer comprising at least one basic nitrogen atom and M is a unit derived from an acid monomer comprising at least one group chosen from carboxylic groups and sulfonic groups; or alternatively

K and M, which are identical or different, are each groups chosen from groups derived from zwitterionic monomers of carboxybetaines and groups derived from zwitterionic monomers of sulfobetaines; or alternatively

K and M, which are identical or different, are each chosen from polymers comprising cationic polymer chains comprising at least one amine group chosen from primary, secondary, tertiary and quaternary amine groups, wherein at least one of the amine groups is substituted with a group chosen from carboxylic groups and sulphonic groups linked via a hydrocarbon radical; or alternatively

K and M form part of a chain of a polymer with an α,β-dicarboxylic ethylene unit wherein one of the carboxylic groups has been caused to react with a polyamine comprising at least one amine group chosen from primary amine groups and secondary amine groups. Representative of the film forming amphoteric polymers defined above that can be used include the following polymers:

(1) The polymers resulting from the copolymerization of a monomer derived from a vinyl compound substituted with a carboxylic group such as acrylic acid, methacrylic acid, maleic acid, and α-chloroacrylic acid, and of a basic monomer derived from a substituted vinyl compound comprising at least one basic atom such as dialkylaminoalkyl methacrylate and acrylate, dialkylaminoalkylmethacrylamide and acrylamide. Such compounds are described in U.S. Pat. No. 3 836 537, the disclosure of which is incorporated by reference herein. Copolymer of the sodium acrylate/acrylamidopropyl-trimethylammonium chloride sold under the name of “POLYQUART KE 3033” by the company HENKEL can also be cited.

The vinyl compound can also be a salt of dialkyldiallylammonium such as diethyldiallylammonium chloride. The copolymers of acrylic acid and the latter monomer are proposed under the name “MERQUAT 280”, “MERQUAT 295” and “MERQUAT PLUS 3330” by the company CALGON.

(2) The polymers comprising units derived from:

a) at least one monomer chosen from acrylamides substituted on the nitrogen by an alkyl radical and methacrylamides substituted on the nitrogen by an alkyl radical,

b) at least one acidic comonomer comprising at least one reactive carboxylic group, and

c) at least one basic comonomer such as comonomers chosen from esters of acrylic acid and esters of methacrylic acid, said esters being substituted with at least one amine chosen from primary, secondary, tertiary and quaternary amines, and the product of quaternization of dimethylaminoethyl methacrylate with a sulfate chosen from dimethyl sulfate and diethyl sulfate.

Some embodiments according to the invention utilize N-substituted acrylamides and methacrylamides comprising (C₂-C₁₂)alkyl groups, such as N-ethylacrylamide, N-tert-butylacrylamide, N-tert-octylacrylamide, N-octylacrylamide, N-decylacrylamide, and N-dodecylacrylamide, as well as the corresponding methacrylamides.

The acidic comonomers can be chosen, for example, from acrylic, methacrylic, crotonic, itaconic, maleic and fumaric acids as well as the (C₁-C₄)alkyl monoesters of entities chosen from maleic anhydride, fumaric anhydride, maleic acid, and fumaric acid.

The basic comonomers can be chosen, for example, from methacrylates of aminoethyl, butylaminoethyl, N,N′-dimethylaminoethyl, and N-tert-butylaminoethyl.

Additionally, the copolymers having the CTFA name (4^{th} edition, 1991) Octylacrylamide/acrylates/butylaminoethylmethacrylate copolymer such as the products sold under the name AMPHOMER and LOVOCRYL 47 by the company NATIONAL STARCH can also be used.

(3) The partially and completely alkylated and crosslinked polyaminoamides derived from polyaminoamides of formula:

[CO—R₁₉CO—Z] (X)

wherein:

R₁₉ is a divalent group chosen from groups derived from saturated dicarboxylic acids, groups derived from dicarboxylic aromatic acids, groups derived from mono- and dicarbocylic aliphatic acids comprising at least one ethylenic double bond, groups derived from an ester of (C₁-C₆)alkanols of said acids, and groups derived from the addition of any one of said aforementioned acids with an amine chosen from bis-primary and bis-secondary amines, and

Z is a divalent group derived from polyalkylene-polyamines chosen from bis-primary, mono- and bis-secondary polyalkylene-polyamines, for example, Z represents:

a) in an amount ranging from 60 mol % to 100 mol %, the group

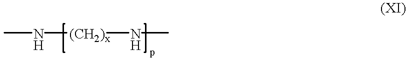

wherein x=2 and p=2 or 3, or alternatively x=3 and p=2,

it being understood that group Z of formula a) is derived from a compound chosen from diethylenetriamine, triethylenetetraamine and dipropylenetriamine;

b) in an amount ranging from 0 mol % to 40 mol %, (1) said groups (Xl) above in which x=2 and p=1 and which said group is derived from a compound chosen from ethylenediamine, and (2) groups derived from piperazine:

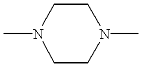

c) in an amount ranging from 0 mol % to 20 mol %, the polyalkylene-polyamine group —NH—(CH₂)₆—NH—, which is derived from hexamethylenediamine, wherein said polyalkylene-polyamine group is crosslinked by adding a bifunctional crosslinking agent (chosen from the epihalohydrins, diepoxides, dianhydrides, and bis-unsaturated derivatives) present in an amount ranging from 0.025 mol to 0.35 mol of crosslinking agent per amine group of the polyamino amide and alkylated by the action of at least one compound chosen from acrylic acid, chloroacetic acid, alkanesultones, and salts thereof.

The saturated dicarboxylic acids are for example chosen from saturated (C₆-C₁₀) dicarboxylic acids such as adipic, 2,2,4-trimethyladipic and 2,4,4-trimethyladipic acid.

Representative dicarboxylic aromatic acids include for example (C₆-C₁₀) dicarboxylic aromatic acids, such as terephthalic acid. And representative mono- and dicarboxylic aliphatic acids comprising at least one ethylenic double bond include for example acrylic, methacrylic and itaconic acids.

The alkanesultones used in the alkylation can, for example, be chosen from propanesultone and butanesultone, and the salts of the alkylating agents can be chosen from sodium and potassium salts of said alkylating agents.

(4) The polymers comprising zwitterionic units of formula:

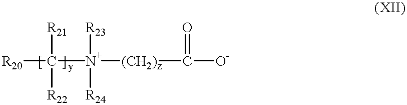

wherein:

R₂₀ is chosen from residues of polymerizable unsaturated groups such as acrylate, methacrylate, acrylamide and methacrylamide groups,

y and z, which can be identical or different, are each chosen from integers ranging from 1 to 3,

R₂₁ and R₂₂, which may be identical or different, are each chosen from hydrogen, and methyl, ethyl and propyl groups,

R₂₃ and R₂₄, which may be identical or different, are each chosen from hydrogen and alkyl groups, provided that the sum of the carbon atoms in R₂₃ and R₂₄ does not exceed 10.

The polymers comprising such units may also comprise units derived from nonzwitterionic monomers such as dimethylaminoethyl acrylate, dimethylaminoethyl methacrylate, diethylaminoethyl acrylate, diethylaminoethyl methacrylate, alkyl acrylates, alkyl methacrylates, alkyl acrylamides, alkyl methacrylamides, and vinyl acetate.

By way of example, there may be mentioned the copolymer of methyl methacrylate/methyl dimethylcarboxymethylammonioethyl methacrylate such as the product sold under the name DIAFORMER Z301 by the company SANDOZ.

(5) The polymers derived from chitosan comprising at least one monomeric unit chosen from formulae (XII), (XIV) and (XV):

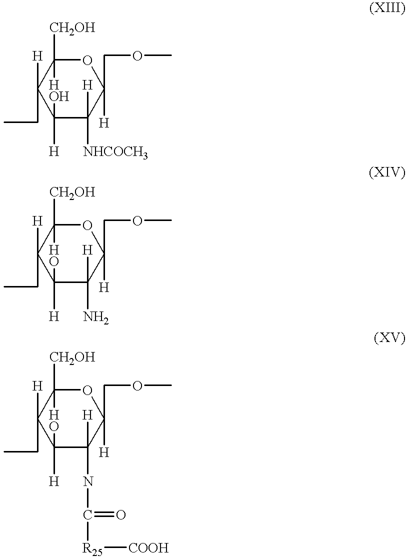

wherein said unit (XIII) is present in an amount ranging for example from 0% to 30%, by weight relative to the total weight of said polymer, said unit (XIV) is present in an amount ranging for example from 5% to 50% by weight relative to the total weight of said polymer, and said unit (XV) is present in an amount ranging for example from 30% to 90% by weight relative to the total weight of said polymer,

and wherein in said unit (XV), R₂₅ is chosen from groups of formula:

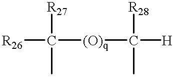

wherein:

q is equal to 0 or 1, and

(i) when q is equal to 0, R₂₆, R₂₇ and R₂₈, which may be identical or different, are each chosen from:

hydrogen,

methyl, hydroxyl, acetoxy, and amino groups,

monoalkylamine and dialkylamine groups optionally interrupted by at least one nitrogen atom and/or optionally substituted with at least one group chosen from amine, hydroxyl, carboxyl, alkylthio and sulfonic groups, and

alkylthio groups wherein said alkyl portion of said alkylthio group carries an amino group,

provided that at least one of said R₂₆, R₂₇ and R₂₈ groups is chosen from hydrogen; and

(ii) when q is equal to 1, R₂₆, R₂₇ and R₂₈, which may be identical or different, are each chosen from hydrogen, and the salts formed by these polymers (5) with bases, and the salts formed by these polymers (5) with acids

(6) The polymers derived from the N-carboxyalkylation of chitosan such as N-carboxymethyl chitosan and N-carboxybutyl chitosan sold under the name “EVALSAN” by the company JAN DEKKER.

(7) The polymers of formula (XVI), which are described for example in French Patent 1 400366, the disclosure of which is incorporated by reference herein:

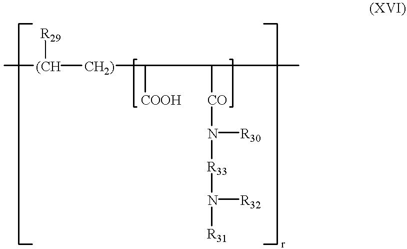

wherein:

r is chosen such that the number-average molecular weight of said polymer ranges from 500 to 6,000,000, such as from 1000 to 1,000,000.

R₂₉ is chosen from hydrogen and CH₃O, CH₃CH₂O, and phenyl groups,

R₃₀ and R₃₁, which are identical or different, are each chosen from hydrogen and lower alkyl groups such as methyl and ethyl,

R₃₂ is chosen from lower alkyl groups such as methyl and ethyl and groups of formula: —R₃₃—N(R₃₁)₂, comprising up to 6 carbon atoms, wherein R₃1 is as defined above and R₃₃ is defined below,

R₃₃ is chosen from —CH₂—CH₂—, —CH₂—CH₂—CH₂—, and —CH₂—CH(CH₃)—.

(8) Amphoteric polymers of the -D-X-D-X-type, which are described for example in U.S. Pat. No. 4,996,059, the disclosure of which is herein incorporated by reference, chosen from:

a) polymers derived from reaction of chloroacetic acid or sodium chloroacetate with at least one compound comprising at least one unit of formula (XVII):

—D—X—D—X—D— (XVII)

wherein D is a group:

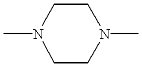

and X is chosen from the symbols E and E′, wherein E and E′, which are identical or different, are each chosen from bivalent groups chosen from alkylene groups comprising at least one chain chosen from linear and branched chains comprising up to 7 carbon atoms in the principal chain, wherein said principal chain is optionally substituted with at least one hydroxyl group, and wherein said alkylene groups optionally comprise at least one atom chosen from oxygen atoms, nitrogen atoms and sulfur atoms, wherein said at least one optional atom is present in the form of at least one group chosen from ether, thioether, sulfoxide, sulfone, sulfonium, alkylamine and alkenylamine groups, and hydroxyl, benzylamine, amine oxide, quaternary ammonium, amide, imide, alcohol, ester and urethane groups, and wherein said alkylene groups optionally comprise 1 to 3 rings chosen from aromatic rings and heterocyclic rings,

b) polymers of formula:

—D—X—D—X— (XIII)

wherein:

D is a group:

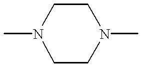

X is chosen from the symbols E and E′ and wherein at least one X is chosen from E′, -E is chosen from bivalent groups chosen from alkylene groups comprising at least one chain chosen from linear and branched chains comprising up to 7 carbon atoms in the principal chain, wherein said principal chain is optionally substituted with at least one hydroxyl group, and wherein said alkylene groups optionally comprise at least one atom chosen from oxygen atoms, nitrogen atoms, and sulfur atoms, wherein said at least one optional atom is present in the form of at least one group chosen from ether, thioether, sulfoxide, sulfone, sulfonium, alkylamine and alkenylamine groups, and hydroxyl, benzylamine, amine oxide, quaternary ammonium, amide, imide, alcohol, ester and urethane groups, and wherein said alkylene groups optionally comprise 1 to 3 rings chosen from aromatic rings and heterocyclic rings, and

E′ is a bivalent group chosen from alkylene groups comprising at least one chain chosen from linear and branched chains comprising up to 7 carbon atoms in the principal chain, wherein said principal chain is optionally substituted with at least one hydroxyl group, and wherein said alkylene groups comprise at least one nitrogen atom substituted with an alkyl chain, wherein said alkyl chain is optionally interrupted by an oxygen atom and, wherein said alkyl chain comprises at least one functional group chosen from carboxyl and hydroxyl functional groups, and wherein said at least one alkyl chain is betainized by reaction with a reactant chosen from chloroacetic acid and sodium chloroacetate.

The amphoteric polymers of family (1) are utilized in certain embodiments of the invention.

According to the invention, the at least one polymer chosen from cationic and amphoteric polymers may be present in an amount ranging for example from 0.01% to 10% by weight relative to the total weight of the composition, such as from 0.05% to 5% by weight relative to the total weight of the composition, and further such as from 0.1% to 3% by weight relative to the total weight of the composition.

Surfactants

The ready-to-use composition according to the invention can comprise at least one surfactant, which is present in at least one of said at least one dyeing composition (A), said at least one oxidizing composition (B), and said at least one dyeing composition (A) and said at least one oxidizing composition (B).

The at least one surfactant may be chosen from anionic, amphoteric, nonionic, zwitterionic and cationic surfactants.

Representative choices for the at least one surfactant include the following:

(i) Anionic Surfactants

Representative anionic surfactants include salts (for example alkaline salts, such as sodium salts, ammonium salts, amine salts, amino alcohol salts and magnesium salts) of the following compounds: alkyl sulfates, alkyl ether sulfates, alkylamidoether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl(C₆-C₂₄) sulfosuccinates, alkyl(C₆-C₂₄) ether sulfosuccinates, alkyl(C₆-C₂₄)amide sulfosuccinates, alkylsulfosuccinamates alkyl(C₆-C₂₄) sulfoacetates, acyl(C₆-C₂₄) sarcosinates, acyl(C₆-C₂₄) glutamates, acyl isethionates, N-acyltaurates, and alkyl(C₆-C₂₄)polyglycoside carboxylic esters such as alkylglucoside citrates, alkylpolyglycoside tartrate, alkylpolyglycoside sulfosuccinates, and alkyl sulfosuccinamates. The alkyl and acyl radicals of all of these various compounds can for example comprise from 12 to 20 carbon atoms, and the aryl radicals can for example be chosen from phenyl and benzyl groups.

For example, anionic surfactants can be chosen from fatty acid salts such as the salts of oleic acid, ricinoleic acid, palmitic acid, stearic acid, the acids of copra oil and the acids of hydrogenated copra oil, and acyl lactylates in which the acyl radical comprises from 8 to 20 carbon atoms. At least one weakly anionic surfactant can also be used, such as alkyl-D-galactosideuronic acids and their salts, as well as polyoxyalkylenated carboxylic (C₆-C₂₄)alkyl ether acids, polyoxyalkylenated carboxylic (C₆-C₂₄)alkylaryl ether acids, polyoxyalkylenated carboxylic (C₆-C₂₄)alkyl amidoether acids and their salts, for example, those comprising from 2 to 50 ethylene oxide groups.

(ii) Nonionic Surfactants:

Useful nonionic surfactants include compounds that are well known per se (see for example in this respect “Handbook of Surfactants” by M. R. Porter, published by Blackie & Son (Glasgow and London), 1991, pp.116-178), the disclosure of which is incorporated by reference herein. Thus, nonionic surfactants can include alcohols, α-diols, and polyethoxylated alkylphenols and polypropoxylated alkylphenols comprising at least one fatty chain comprising for example from 8 to 18 carbon atoms, wherein the number of ethylene oxide and propylene oxide groups can range for example from 2 to 50.

Additionally, copolymers of ethylene oxide, copolymers of propylene oxide, condensates of ethylene oxide with fatty alcohols, condensates of propylene oxide with fatty alcohols, polyethoxylated fatty amides, such as those comprising from 2 to 30 mol of ethylene oxide, polyglycerolated fatty amides on average comprising 1 to 5 glycerol groups, such as from 1.5 to 4, polyethoxylated fatty amines comprising for example from 2 to 30 mol of ethylene oxide, oxyethylenated fatty acid esters of sorbitan comprising from 2 to 30 mol of ethylene oxide, fatty acid esters of sucrose, fatty acid esters of polyethylene glycol, alkylpolyglycosides, N-alkylglucamine derivatives, amine oxides, such as the oxides of (C₁₀-C₁₄) alkylamines, and N-acylaminopropylmorpholine oxides can also be used. It will be noted that the alkylpolyglycosides are nonionic surfactants that can be suitable in the context of the present invention.

(iii) Amphoteric or Zwitterionic Surfactants:

Representative amphoteric and zwitterionic surfactants can be chosen from aliphatic secondary and tertiary amine derivatives in which the aliphatic radical is chosen from linear and branched chain radicals comprising 8 to 18 carbon atoms and comprising at least one water-soluble anionic group (chosen for example from carboxylate, sulfonate, sulfate, phosphate and phosphonate); mention may also be made of (C₈-C₂₀)alkylbetaines, sulfobetaines, (C₈-C₂₀)alkylamido(C₁-C₆)alkylbetaines and (C₈-C₂₀)alkylamido(C₁-C₆)alkylsulfobetaines.

Representative amine derivatives include the products sold under the name MIRANOL, as described in U.S. Pat. Nos. 2,528,378 and 2,781,354, the disclosures of which are incorporated by reference herein, and classified in the CTFA dictionary, 3^{rd} edition, 1982, under the names Amphocarboxyglycinates and Amphocarboxypropionates, having the respective structures:

R₃₄—CONHCH₂CH₂—N⁺(R₃₅)(R₃₆)(CH₂COO—)

wherein:

R₃₄ is chosen from alkyl groups derived from an acid R₂—COOH present in hydrolysed copra oil, and heptyl, nonyl and undecyl radicals,

R₃₅ is a beta-hydroxyethyl group, and R₃₆ is a carboxymethyl group; and

R₃₄′—CONHCH₂CH₂—N(B)(C)

wherein:

(B) is —CH₂CH₂OX′, wherein X′ is an entity chosen from a —CH₂CH₂—COOH group and a hydrogen atom,

(C) is —(CH₂)_{z}—Y′, wherein z=1 or 2, and wherein Y′ is an entity chosen from —COOH and —CH₂—CHOH—SO₃H groups,

R₃₄′ is chosen from alkyl groups, such as (a) alkyl groups of an acid R₅—COOH present in oils chosen from copra oil and hydrolysed linseed oil, (b) alkyl groups, such as C₇, C₉, C₁₁ and C₁₃ alkyl groups, and (c) C₁₇ alkyl groups and the iso forms, and unsaturated C₁₇ groups.

Such representative compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

By way of example, mention may be made of the cocoamphodiacetate sold under the trade name MIRANOL C2M Concentrate by the company RHODIA CHIMIE.

(iv) Cationic Surfactants:

Representative cationic surfactants include salts of optionally polyoxyalkylenated primary, secondary and tertiary fatty amines; quaternary ammonium salts such as tetraalkylammonium, alkylamidoalkyltrialkylammonium, trialkylbenzylammonium, and trialkylhydroxyalkylammonium, alkylpyridinium chlorides, alkylpyridinium bromides, imidazoline derivatives; and amine oxides of cationic nature.

The at least one surfactant may be present in the composition according to the invention generally in an amount ranging for example from 0.01% to 40% by weight relative to the total weight of the composition, such as from 0.1% to 30% by weight relative to the total weight of the composition.

The reducing agents which may be used to preserve the oxidation dyes may be present in a composition according to the invention in an amount ranging, for example, from 0.05 to 1.5% by weight relative to the total weight of the composition. Non-limiting examples of said reducing agents can be chosen from sodium sulfite, thioglycolic acid, thiolactic acid, sodium bisulfite, dihydroascorbic acid, hydroquinone, 2-methylhydroquinone, tert-butylhydroquinone and homogentisic acid. In one embodiment of the invention, said reducing agents may also be chosen from N-acetyl-L-cysteine described in French patent application, FR-9903829, a ketose, and a ketohexose such as, fructose as described in French patent application, FR-9904338, all of the above references are incorporated by reference herein.

One skilled in the art should take care to select said optionally complementary compounds, such that the advantageous properties intrinsically associated with the dye composition according to the invention are not, or are not substantially, adversely affected by the additions envisaged.

The dyeing composition according to the invention may be provided in various forms, such as, liquids, powders, creams, gels, optionally pressurized, or in any other form suitable for dyeing keratinous fibers, such as human hair.

According to the invention, when said at least one oxidation dye precursor chosen from the 1-(4-aminophenyl)pyrrolidines of formula (I) and at least one 2 electron oxidoreductase or at least one 4 electron oxidoreductase are present in the same ready-to-use composition, the ready-to-use composition should be free of gaseous oxygen to minimize any premature oxidation of the 1-(4-aminophenyl)pyrrolidine(s) of formula (I).

The invention also relates to a method for dyeing keratinous fibers, for example, human keratinous fibers such as hair, comprising:

(1) applying to said fibers at least one ready-to-use dyeing composition comprising, in a medium suitable for dyeing:

(i) at least one enzymatic oxidizing system comprising at least one enzyme chosen from 2 electron oxidoreductases, 4 electron oxidoreductases, and peroxidases; and

(ii) at least one oxidation dye precursor chosen from 1-(4-aminophenyl)-pyrrolidines of formula (I) and acid addition salts thereof:

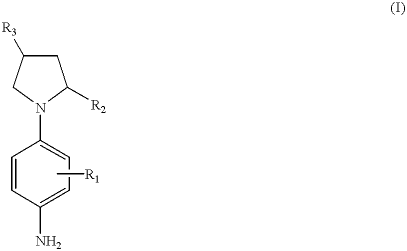

wherein:

R₁ is chosen from a hydrogen atom, (C₁-C₆)alkyl groups, (C₁-C₅)monohydroxyalkyl groups, and (C₂-C₅)polyhydroxyalkyl groups;

R₂ is chosen from a hydrogen atom, a —CONH₂ group, (C₁-C₅)monohydroxyalkyl groups, and (C₂-C₅)polyhydroxyalkyl groups;

R₃ is chosen from a hydrogen atom, and a hydroxyl group;

(2) developing a color; and

(3) rinsing said fibers and optionally shampooing and optionally further rinsing said fibers; and

(4) drying said fibers.

In one embodiment of the invention, the application temperature ranges, for example, from room temperature to 80° C., such as, for example, from room temperature to 60° C., and further still, from 35° C. to 50° C.

The time sufficient for developing the color on the keratinous fibers generally ranges, for example, from 1 to 60 minutes, such as, 5 to 30 minutes.

Another embodiment of the invention, is a method for dyeing keratinous fibers, for example, human keratinous fibers such as hair, comprising:

(1) storing a composition (A) comprising, in a medium suitable for dyeing:

(i) at least one enzymatic oxidizing system comprising at least one enzyme chosen from 2 electron oxidoreductases, 4 electron oxidoreductases, and peroxidases;

(2) storing, separately from said composition (A), a composition (B) comprising, in a medium suitable for dyeing:

(ii) at least one oxidation dye precursor chosen from 1-(4-aminophenyl)pyrrolidines of formula (I) and acid addition salts thereof:

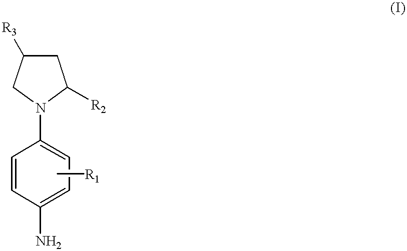

wherein:

R₁ is chosen from a hydrogen atom, (C₁-C₆)alkyl groups, (C₁-C₅)monohydroxyalkyl groups, and (C₂-C₅)polyhydroxyalkyl groups;

R₂ is chosen from a hydrogen atom, a —CONH₂ group, (C₁-C₅)monohydroxyalkyl groups, and (C₂-C₅)polyhydroxyalkyl groups;

R₃ is chosen from a hydrogen atom, and a hydroxyl group;

(3) combining said composition (A) and said composition (B) together just prior to use, to produce a mixture;

(4) applying said mixture to said fibers;

(5) developing a color; and

(6) rinsing said fibers and optionally shampooing and optionally further rinsing said fibers; and

(7) drying said fibers.

Another embodiment of the invention is a multi-compartment dyeing device, dyeing “kit” for oxidation dyeing keratinous fibers, for example, human keratinous fibers, such as hair. This device may be equipped with a means to deliver the desired composition of the invention to said fibers. A non-limiting example of this device is disclosed in French patent FR-2,586,913, incorporated by reference herein.

Another embodiment of the invention, is a kit comprising at least two compartments, wherein:

(1) a first compartment comprises, in a medium suitable for dyeing:

at least one enzymatic oxidizing system comprising at least one enzyme chosen from 2 electron oxidoreductases, 4 electron oxidoreductases, and peroxidases; and

(2) a second compartment comprises, in a medium suitable for dyeing:

at least one oxidation dye precursor chosen from 1-(4-aminophenyl)pyrrolidines of formula (I) and acid addition salts thereof:

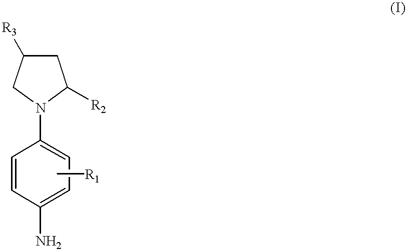

wherein:

R₁ is chosen from a hydrogen atom, (C₁-C₆)alkyl groups, (C₁-C₅)monohydroxyalkyl groups, and (C₂-C₅)polyhydroxyalkyl groups;

R₂ is chosen from a hydrogen atom, a —CONH₂ group, (C₁-C₅)monohydroxyalkyl groups, and (C₂-C₅)polyhydroxyalkyl groups;

R₃ is chosen from a hydrogen atom, and a hydroxyl group.

Unless otherwise indicated, all numbers expressing quantities of ingredients, properties such as molecular weight, reaction conditions, and so forth used in the specification and claims are to be understood as being modified in all instances by the term “about”. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The following examples are intended to illustrate the invention without in anyway limiting the scope thereof.

### EXAMPLE 1

A ready-to-use dyeing composition A according to the invention containing, as oxidation dye precursor, 1-(4-aminophenyl)pyrrolidine dihydrochloride, as coupler, 1-β-hydroxyethyloxy-2,4-diaminobenzene and, as oxidant, uricase (oxidase containing 2 electrons) was compared with a prior art composition B containing, as oxidation dye precursor, N,N-bis(β-hydroxyethyl)-para-phenylenediamine sulfate.

The compositions are given below.

Each of composition was applied to locks of natural grey hair comprising 90% white fibers, for 30 minutes at 40° C.

At the end of the exposure time, the hair locks were rinsed, washed with a shampoo and then dried.

The color was then measured with a MINOLTA CM2002 colorimeter in the L*a*b* system.

In the L*a*b* system, the 3 parameters denote respectively the intensity (L*), the shade (a*) and the saturation (b*).

According to this system, the higher the value of L, the lighter and less intense the color. Conversely, the lower the value of L, the deeper or more intense the color.

a* and b* indicate two axes of colors, a* indicates the green/red color axis and b* the blue/yellow color axis. Values close to zero for a* and b* correspond to grey shades.

The selectivity of the color AE may be calculated by applying the following equation:

ΔE=\sqrt{(L*−L₀*)²+(a*−a₀*)²+(b*−b₀*)²}

In this equation, ΔE represents the difference in color between two locks, (in the present case the selectivity of the color), L*, a* and b* represent the intensity, the shade and the saturation of the lock of natural hair dyed, respectively. L₀*, a₀* and b₀* represent the intensity, the shade and the saturation of the lock of permanently waved hair dyed, respectively.

The higher the value of ΔE, the greater the difference in color between the two locks, and in the present case, the greater the selectivity of the dyeing.

The results have been grouped together in Table (I) below.

| | |
|---|---|
| Composition A (invention) | |
| 1-(4-Aminophenyl)pyrrolidine dihydrochloride | 0.705 g |
| 1-β-Hydroxyethyloxy-2,4-diaminobenzene | 0.723 g |
| dihydrochloride | |
| N-Acetyl-L-cysteine | 0.10 g |
| Uricase | 20 × 10³ U units |
| Uric acid | 1 g |
| Polyglycerol monooleate containing 10 mol of glycerol | 1 g |
| Aculyn 22 (Rohm and Haas) | 0.75 g AS* |
| 2-Amino-2-methyl-1-propanol | qs pH 9.5 |
| Demineralized water qs | 100 g |
| Composition B (prior art) | |
| N,N-bis(β-Hydroxyethyl)-para-phenylenediamine | 0.588 g |
| sulfate | |
| 1-β-Hydroxyethyloxy-2,4-diaminobenzene | 0.723 g |
| dihydrochloride | |
| N-Acetyl-L-cysteine | 0.10 g |
| Uricase | 20 × 10³ U units |
| Uric acid | 1 g |
| Polyglycerol monooleate containing 10 mol of glycerol | 1 g |
| Aculyn 22 (Rohm and Haas) | 0.75 g AS* |
| 2-Amino-2-methyl-1-propanol | qs pH 9.5 |
| Demineralized water qs | 100 g |

*denotes Active substance

TABLE (I)

| | | | | Dyeing |
|---|---|---|---|---|
| | | | | selectivity |
| Composition | L* | a* | b* | ΔE |
| A (invention) | | | | |
| Natural hair | 23.07 | −1.35 | −11.47 | 3.02 |
| Permanently waved hair | 21.27 | −0.48 | −9.21 | |
| B (prior art) | | | | |
| Natural hair | 35.62 | −5.53 | −7.37 | 11.61 |
| Permanently waved hair | 25.10 | −2.27 | −11.04 | |

These results illustrate that dyeing according to the invention (A) is more intense (lower L*) and less selective (smaller ΔE) than that of the prior art (B).

### EXAMPLE 2

A ready-to-use dyeing composition A according to the invention containing, as oxidation dye precursor, 1-(4-aminophenyl)pyrrolidine dihydrochloride, as coupler, 1-β-hydroxyethyloxy-2,4-diaminobenzene and, as oxidant, a laccase (oxidase containing 4 electrons) was compared with a prior art composition B containing, as oxidation dye precursor, N,N-bis(β-hydroxyethyl)-para-phenylenediamine sulfate. The compositions are given below.

Each of the compositions was applied to locks of natural grey hair comprising 90% white, for 30 minutes at 40° C.

At the end of the exposure time, the hair locks were rinsed, washed with a shampoo and then dried.

The results have been grouped together in Table (II) below.

| Composition A (invention) | |
|---|---|
| [00281] | |
| 1-(4-Aminophenyl)pyrrolidine dihydrochloride | 0.705 g |
| 1-β-Hydroxyethyloxy-2,4-diaminobenzene dihydro- | 0.723 g |
| chloride | |
| Carboxymethylchitosan (OLEVASAN from | 2 g AS* |
| the company SINOLION) | |
| Laccase | 30 × 10⁶ U units |
| Citric acid | qs pH 7 |
| Demineralized water qs | 100 g |
| Composition B (prior art) | |
| [00282] | |
| N,N-bis(β-Hydroxyethyl)-para-phenylenediamine | 0.588 g |
| sulfate | |
| 1-β-Hydroxyethyloxy-2,4-diaminobenzene dihydro- | 0.723 g |
| chloride | |
| Carboxymethylchitosan (OLEVASAN from the | 2 g AS* |
| company SINOLION) | |
| Laccase | 30 × 10⁶ U units |
| Citric acid | qs pH 7 |
| Demineralized water qs | 100 g |

*denotes Active substance

TABLE (I)

| Composition | L* |
|---|---|
| A (invention) | |
| Natural hair . . . → | 30.62 |
| Permanently waved hair → | 22.28 |
| B (prior art) | |
| Natural hair → | 37.23 |
| Permanently waved hair . . . → | 32.31 |

These results demonstrate that the dyeing according to the invention (A) is more intense (lower L*) than that of the prior art (B).

What is claimed:

1. A ready-to-use composition for oxidation dyeing of keratinous fibers comprising, in a medium suitable for dyeing:
(1) at least one enzymatic oxidizing system comprising at least one enzyme chosen from 2 electron oxidoreductases, 4 electron oxidoreductases, and peroxidases; and
(2) at least one oxidation dye precursor chosen from 1-(4-aminophenyl)pyrrolidines of formula (I) and acid addition salts thereof: 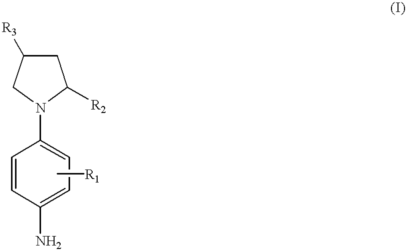
wherein:
R₁ is chosen from a hydrogen atom, (C₁-C₆)alkyl groups, (C₁-C₅)monohydroxyalkyl groups, and (C₂-C₅)polyhydroxyalkyl groups;
R₂ is chosen from a hydrogen atom, a —CONH₂ group, (C₁-C₅)monohydroxyalkyl groups, and (C₂-C₅)polyhydroxyalkyl groups;
R₃ is chosen from a hydrogen atom, and a hydroxyl group.

2. A composition according to claim 1, wherein said R₁, said R₂ and said R₃ are each a hydrogen atom.

3. A composition according to claim 1, wherein said R₁ and said R₃ are each a hydrogen atom and said R₂ is a —CH₂OH group.

4. A composition according to claim 1, wherein said R₁ is a hydrogen atom, said R₂ is a —CH₂OH group and said R₃ is a hydroxyl group.

5. A composition according to claim 1, wherein said R₁ and said R₃ are each a hydrogen atom and said R₂ is a —CONH₂ group.

6. A composition according to claim 1, wherein said acid addition salts are chosen from hydrochlorides, hydrobromides, sulphates, tartrates, lactates and acetates.

7. A composition according to claim 1, wherein said at least one oxidation dye precursor chosen from the 1-(4-aminophenyl)pyrrolidines of formula (I) and acid addition salts thereof is present in said composition in an amount ranging from 0.001 to 10% by weight relative to the total weight of said composition.

8. A composition according to claim 7, wherein said at least one oxidation dye precursor chosen from the 1-(4-aminophenyl)pyrrolidines of formula (I) and acid addition salts thereof is present in said composition in an amount ranging from 0.01 to 8% by weight of the total weight of said composition.

9. A composition according to claim 1, further comprising at least one corresponding donor when said 2 electron oxidoreductases are present in said composition, wherein said 2 electron oxidoreductases are chosen from pyranose oxidases, glucose oxidases, glycerol oxidases, lactate oxidases, pyruvate oxidases, uricases, choline oxidases, sarcosine oxidases, bilirubin oxidases and amino acid oxidases.

10. A composition according to claim 1, wherein said 2 electron oxidoreductases are present from 0.01 to 20% by weight relative to the total weight of said composition.

11. A composition according to claim 10, wherein said 2 electron oxidoreductases are present from 0.1 to 5% by weight relative to the total weight of said composition.

12. A composition according to claim 1, wherein said 4 electron oxidoreductases are chosen from laccases, tyrosinases, catechol oxidases and polyphenol oxidases.

13. A composition according to claim 12, wherein said laccases are chosen from laccases of plant, laccases of animal origin, laccases of fungal origin, laccases of bacterial origin and laccases of biotechnological origin.

14. A composition according to claim 13, wherein said laccases of plant origin are chosen from laccases extracted from Anacardiaceae, Podocarpaceae, Rosmarinus off., Solanum tuberosum, Iris sp., Coffea sp., Daucus carrota, Vinca minor, Persea americana, Catharenthus roseus, Musa sp., Malus pumila, Gingko biloba, Monotropa hypopithys, Aesculus sp., Acer pseudoplatanus, Prunus persica and Pistacia palaestina.

15. A composition according to claim 13, wherein said laccases are chosen from plant origin and biotechnological origin.

16. A composition according to claim 15, wherein said laccases are chosen from the laccases extracted from Polyporus versicolor, Rhizoctonia praticola, Rhus vernicifera, Scytalidium, Polyporus pinsitus, Myceliophtora thermophila, Rhizoctonia solani, Pyricularia oryzae, Trametes versicolor, Fomes fomentarius, Chaetomium thermophile, Neurospora crassa, Colorius versicol, Botrytis cinerea, Rigidoporus lignosus, Phellinus noxius, Pleurotus ostreatus, Aspergillus nidulans, Podospora anserina, Agaricus bisporus, Ganoderma lucidum, Glomerella cingulata, Lactarius piperatus, Russula delica, Heterobasidion annosum, Thelephora terrestris, Cladosporium cladosporioides, Cerrena unicolor, Coriolus hirsutus, Ceriporiopsis subvermispora, Coprinus cinereus, Panaeolus papilionaceus, Panaeolus sphinctrinus, Schizophyllum commune, Dichomitius squalens, and variants thereof.

17. A composition according to claim 1, wherein said 4 electron oxidoreductases are present from 0.01 to 20% by weight relative to the total weight of said composition.

18. A composition according to claim 17, wherein said 4 electron oxidoreductases are present from 0.1 to 5% by weight relative to the total weight of said composition.

19. A composition according to claim 1, wherein said peroxidases are chosen from simplex peroxidases and catalases.

20. A composition according to claim 1, further comprising at least one corresponding donor when said peroxidases are present in said composition, wherein said peroxidases are chosen from NADH peroxidases, fatty acid peroxidases, NADPH peroxidases, cytochrome c peroxidases, iodide peroxidases, chloride peroxidases, L-ascorbates and glutathione peroxidases.

21. A composition according to claims 1, wherein said peroxidases are chosen from peroxidases of plant, peroxidases of animal, peroxidases of fungal, peroxidases of bacterial and peroxidases of biotechnological origin.

22. A composition according to claim 21, wherein said peroxidases are chosen from peroxidases obtained from apple, apricot, barley, black radish, beet, cabbage, carrot, corn, cotton, garlic, grape, mint, rhubarb, soy bean, spinach, ink cap, bovine milk, Acetobacter peroxidans microorganisms, Staphylococcus faecalis microorganisms and Arthromyces ramosus microorganisms.

23. A composition according to claim 1, wherein said peroxidases are present from 0.0001 to 20% by weight relative to the total weight of said composition.

24. A composition according to claim 23, wherein said peroxidases are pre sent from 0.001 to 10% by weight relative to the total weight of said composition.

25. A composition according to claim 1, further comprising at least one coupler chosen from meta-aminophenols, meta-phenylenediamines, meta-diphenols, naphthols, heterocyclic couplers, and acid addition salts of any of the foregoing compounds.

26. A composition according to claim 25, wherein said at least one coupler is chosen from 2,4-diamino-1-(β-hydroxyethyloxy)benzene, 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2-amino-4-(β-hydroxyethylamino)-1-methoxybenzene, 1,3-diaminobenzene, 1,3-bis-(2,4-diaminophenoxy)propane, sesamol, 1-amino-2-methoxy-4,5-methylenedioxybenzene, a-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, 2-amino-3-hydroxypyridine, 3,6-dimethylpyrazolo[3,2-c]-1,2,4-triazole, 2,6-dimethylpyrazolo[1,5-b]-1,2,4-triazole and acid addition salts of any of the foregoing compounds.

27. A composition according to claim 25, said at least one coupler is present in an amount ranging from 0.0001 to 15% by weight relative to the total weight of said composition.

28. A composition according to claim 1, further comprising at least one direct dye chosen from chosen from neutral, cationic, and anionic nitro dyes, cationic, and anionic azo dyes and cationic, and anionic anthraquinone dyes.

29. A composition according to claim 28, wherein said at least one direct dye is present in an amount ranging from 0.001 to 20% relative to the total weight of the composition.

30. A composition according to claim 29, wherein said at least one direct dye is present in an amount ranging from 0.01 to 10% relative to the total weight of the composition.

31. A composition according to claim 1, wherein said medium suitable for dyeing is chosen from water and a mixture of water and at least one organic solvent.

32. A composition according to claim 31, wherein said at least one organic solvent is present in an amount ranging from 1 to 40% by weight relative to the total weight of said composition.

33. A composition according to claim 1, further comprising at least one nonionic surfactant present in an amount of at least 0.01% by weight relative to the total weight of said composition.

34. A composition according to claim 1, further comprising at least one reducing agent present in an amount of at least 0.05% by weight relative to the total weight of said composition.

35. A composition according to claim 1, further comprising at least one polymer chosen from cationic polymers and amphoteric polymers.

36. A composition according to claim 35, wherein said at least one polymer is present in an amount of at least 0.01% by weight relative to the total weight of said composition.

37. A composition according to claim 1, wherein said composition has a pH ranging from 3to 11.

38. A composition according to claim 37, wherein said composition has a pH ranging from 4to 9.

39. A composition according to claim 1, further comprising at least one agent for adjusting pH chosen from acidifying agents and alkalinizing agents.

40. A composition according to claim 1, wherein said composition is chosen from a liquid, a cream, a gel, and any other form appropriate for dyeing keratinous fibers, further wherein said composition is optionally pressurized.

41. A method for dyeing keratinous fibers comprising:
(1) applying to said fibers at least one ready-to-use dyeing composition comprising, in a medium suitable for dyeing:
(i) at least one enzymatic oxidizing system comprising at least one enzyme chosen from 2 electron oxidoreductases, 4 electron oxidoreductases, and peroxidases; and
(ii) at least one oxidation dye precursor chosen from 1-(4-aminophenyl)pyrrolidines of formula (I) and acid addition salts thereof: 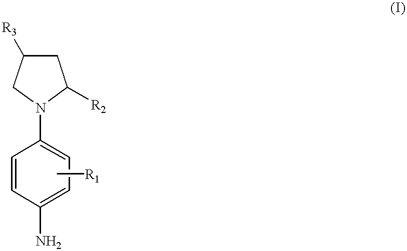
wherein:
R₁ is chosen from a hydrogen atom, (C₁-C₆)alkyl groups, (C₁-C₅)monohydroxyalkyl groups, and (C₂-C₅)polyhydroxyalkyl groups;
R₂ is chosen from a hydrogen atom, a —CONH₂ group, (C₁-C₅)monohydroxyalkyl groups, and (C₂-C₅)polyhydroxyalkyl groups;
R₃ is chosen from a hydrogen atom, and a hydroxyl group,;
(2) developing a color; and
(3) rinsing said fibers and optionally shampooing and optionally further rinsing said fibers; and
(4) drying said fibers, wherein said method has an application temperature ranging from 20-80° C.

42. A method according to claim 41, wherein said application temperature ranges from 20 and 50° C.

43. A method according to claim 41, wherein the time for developing said color ranges from 1 to 60 minutes.

44. A method according to claim 43, wherein the time for developing said color ranges from 5 to 30 minutes.

45. A method for dyeing keratinous fibers comprising:
(1) storing a composition (A) comprising, in a medium suitable for dyeing:
(i) at least one enzymatic oxidizing system comprising at least one enzyme chosen from 2 electron oxidoreductases, 4 electron oxidoreductases, and peroxidases;
(2) storing, separately from said composition (A), a composition (B) comprising, in a medium suitable for dyeing:
(ii) at least one oxidation dye precursor chosen from 1-(4-aminophenyl)pyrrolidines of formula (I) and acid addition salts thereof: 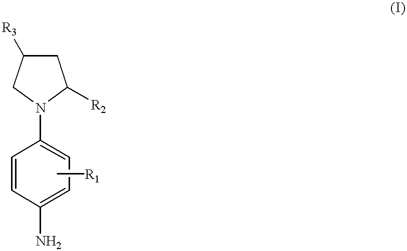
wherein:
R₁ is chosen from a hydrogen atom, (C₁-C₆)alkyl groups, (C₁-C₅)monohydroxyalkyl groups, and (C₂-C₅)polyhydroxyalkyl groups;
R₂ is chosen from a hydrogen atom, a —CONH₂ group, (C₁-C₅)monohydroxyalkyl groups, and (C₂-C₅)polyhydroxyalkyl groups;
R₃ is chosen from a hydrogen atom, and a hydroxyl group;
(3) combining said composition (A) and said composition (B) together just prior to use, to produce a mixture;
(4) applying said mixture to said fibers;
(5) developing a color; and
(6) rinsing said fibers and optionally shampooing and optionally further rinsing said fibers; and
(7) drying said fibers.

46. A kit comprising at least two compartments, wherein:
(1) a first compartment comprises, in a medium suitable for dyeing:
at least one enzymatic oxidizing system comprising: at least one enzyme chosen from 2 electron oxidoreductases, 4 electron oxidoreductases, and peroxidases; and
(2) a second compartment comprises, in a medium suitable for dyeing:
at least one oxidation dye precursor chosen from 1-(4-aminophenyl)pyrrolidines of formula (I) and acid addition salts thereof: 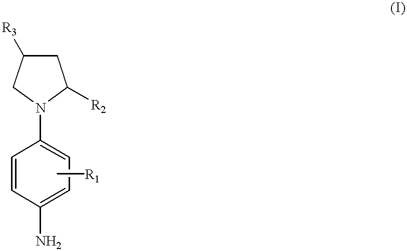
wherein:
R₁ is chosen from a hydrogen atom, (C₁-C₆)alkyl groups, (C₁-C₅)monohydroxyalkyl groups, and (C₂-C₅)polyhydroxyalkyl groups;
R₂ is chosen from a hydrogen atom, a —CONH₂ group, (C₁-C₅)monohydroxyalkyl groups, and (C₂-C₅)polyhydroxyalkyl groups;
R₃ is chosen from a hydrogen atom, and a hydroxyl group.

47. A composition according to claim 1, further comprising at least one additional oxidation base provided that said at least one additional oxidation base is other than said at least one oxidation precursor of formula (I).

48. A composition according to claim 47, wherein said at least one additional oxidation base is presenting an amount ranging from 0.001 to 15% by weight relative to the total weight of said composition.
